(19) United States
(12) Patent Application Publication
(10) Pub. No.: US 2010/0215744 A1
(43) Pub. Date: Aug. 26, 2010

(54) Hydroxypropyl Substitution Used to Regulate Dissolution of a Chemical

(75) Inventors: David Watt, Scottsdale, AZ (US); Steven B. Newhard, Scottsdale, AZ (US)

Correspondence Address: OHLANDT, GREELEY, RUGGIERO & PERLE,LLP;MEDICIS PHARMACEUTICAL CORPORATION, ONE LANDMARK SQUARE, 10TH FLOOR, STAMFORD, CT, 06901-2682, US

(73) Assignee: MEDICIS PHARMACEUTICAL CORPORATION

(21) Appl. No.: 12/756,962

(22) Filed: Apr. 8, 2010

Related U.S. Application Data
(63) Continuation-in-part of application No. 12/536,359, filed on Aug. 5, 2009.
(60) Provisional application No. 61/086,728, filed on Aug. 6, 2008.
(60) Provisional application No. 61/210,882, filed on Mar. 23, 2009.

(51) Int. Cl. A61K 9/22; A61K 31/65; A61P 43/00

(52) U.S. Cl. 424/468; 514/152

(57) ABSTRACT

A method of producing a batch of a tetracycline-class component HPMC extended release pharmaceutical product having a desired dissolution profile, comprising: selecting a dissolution rate-controlling polymer comprising an HPMC component, the HPMC component having a selected % HP value; validating that the % HP in the selected HPMC component is such that a mean sample of the product complies with the desired dissolution profile over each time point in the dissolution profile, and preparing the product by preparing a formulation comprising a pharmaceutically effective amount of the tetracycline-class chemical and the selected HPMC component with the % HP value. There is also provided a method of predicting the dissolution rate profile over a number of dosage forms.

### CROSS REFERENCE TO RELATED APPLICATIONS

This application is a continuation in part of U.S. patent application Ser. No. 12/536,359, filed on Aug. 5, 2009, which claims the benefit of U.S. Provisional Application Ser. No. 61/086,728, filed Aug. 6, 2008, the contents of which are incorporated by reference therein, and which also claims the benefit of U.S. Provisional Application Ser. No. 61/210,882, filed Mar. 23, 2009, the contents of which are also incorporated by reference therein.

### BACKGROUND OF THE DISCLOSURE

1. Field of the Disclosure

The present disclosure relates to methods of predicting, assessing, and/or regulating (including manufacturing) dissolution rates of a chemical (such as water-soluble pharmaceutical active or drug) and related compositions.

2. Background of the Disclosure

Hydroxypropyl methylcellulose (Hypromellose or HPMC) is a polymer that modulates active, such as a drug, release from a hydrophilic swellable matrix tablet. Normally, the drug release rate depends on several formulation variables, such as the concentration and water solubility of the drug. For numerous reasons, including that of product safety and efficacy, achieving consistent release rates of an active or drug is advantageous.

### SUMMARY OF THE DISCLOSURE

The present disclosure provides methods to consistently predict, assess and/or regulate (manufacture) the release rate of a water-soluble chemical, such as water-soluble pharmaceutical active, over a number of HPMC-containing dosage forms.

The present disclosure also provides that regulating, in a certain manner, the percent of hydroxypropoxyl substitution in HPMC (“% HP”) has a significant role in regulating the selected dissolution rate range of an active, or active ingredient in a hydrophilic swellable matrix tablet comprising HPMC for all dosage forms resulting from a batch.

The present disclosure, in another aspect, provides two or more compositions from a batch in which each composition comprises HPMC and the water-soluble chemical. The compositions are manufactured and/or selected to have an average % HP. The % HP is determined in order to impart a level of similarity, in terms of dissolution of the water-soluble chemical, in the batch, to one or more selected standard dissolution time points, such as 1 hr, 2 hr, and 4 hr. time points, or dissolution profile, and/or conditions. In other words, the % HP in the batch is selected to ensure that the dissolution rate of the water-soluble chemical at all time points in a system (such as a high pH system, simulating conditions in the stomach or intestine) are within a high level of similarity to the desired standard/target dissolution profile. In an exemplary embodiment, the water-soluble chemical agent is a water-soluble pharmaceutical active.

The present disclosure further provides methods to predict the dissolution rate of the water-soluble chemical across a number of batches with each batch having a different dosage form.

### BRIEF DESCRIPTION OF THE SEVERAL VIEWS OF THE DRAWINGS

FIG. 1 is a dissolution profile of 45 mg Tablets using a 8.3% HP HPMC.

FIG. 2 is a dissolution profile of 45 mg Tablets using a 8.3% HP HPMC vs. NDA batches (8.3-8.7% HP)

FIG. 3 is a dissolution profile of 45 mg Tablets using a 8.4% HP HPMC.

FIG. 4 is a dissolution profile of 45 mg Tablets showing the effects of % HP on the dissolution profile.

FIG. 5 is a dissolution profile of 90 mg NDA Tablets.

FIG. 6 is a dissolution profile of 90 mg Tablets using a 8.9% HP HPMC.

FIG. 7 is a dissolution profile of 90 mg Tablets showing the effects of HP on the dissolution profile.

FIG. 8 is a dissolution profile of 135 mg NDA Tablets.

FIG. 9 is a dissolution profile of 135 mg Tablets showing the effects of HP on the dissolution profile.

FIG. 10 is a dissolution profile of 135 mg Tablets showing the effects of HP on the dissolution profile.

FIG. 11 is a dissolution profile of 65 mg Tablets.

FIG. 12 is a dissolution profile of 115 mg Tablets.

FIG. 13 is a comparison of the dissolution profiles of 65 mg and 115 mg Tablets.

FIG. 14 is a dissolution profile of 115 mg Tablets.

FIG. 15 is a dissolution profile of 90 mg ER Tablets.

FIG. 16 is a comparison of dissolution profiles of 135 mg ER tablets.

FIG. 17 is a comparison of dissolution profiles of 45 mg ER tablets.

FIG. 18 is a comparison of dissolution profiles of 90 mg ER tablets.

FIG. 19 is a comparison of dissolution profiles of 65 mg ER tablets.

FIG. 20 is a comparison dissolution profiles of 115 mg ER tablets.

### DETAILED DESCRIPTION OF THE DISCLOSURE

In one aspect, the present disclosure is directed to methods for consistently assessing and/or regulating and/or predicting a desired dissolution profile or dissolution rates at several time points within a system of a water-soluble chemical, such as an active, that may be, for example, a pharmaceutical active or a drug, in a hydrophilic swellable matrix tablet including hydroxypropyl methylcellulose (HPMC).

As used herein, dosage form can mean a different form of a dosage, such as a tablet or capsule, as well as a different strength.

Prior to the present disclosure, it was thought that various other factors played the determinative role in attempts to make the dissolution rate of an active meet a standard/target profile. For example, it was thought that particle size, hardness of the tablet, and the properties of various tablet coatings primarily affected the dissolution rate of the active from the tablet.

However, it was only discovered unexpectedly that the percent of hydroxypropoxyl substitution in HPMC plays a significant, direct, and frequently determinative role in the dissolution rate of the active in a hydrophilic swellable matrix tablet having a significant (greater than 20% of the total weight of the dosage form) amount of HPMC, which, generally speaking, is any amount in which the % HP is the dominant factor in terms of the dissolution rate of the active. The significant amount of HPMC means that the dosage form has at least 20% to 30% HPMC. Unless otherwise stated, the dosage forms in this disclosure include this significant amount of HPMC and references to HPMC dosage forms mean HPMC formulations including such an amount of HPMC.

Given this understanding, it is conceived that % HP will similarly act as a determinative dissolution rate factor over a spectrum or range of dosage forms and in a variety of HPMC formulations (for example, capsules, caplets, tablets and the like.

This unexpected and surprising revelation allows for the prediction within an accuracy of discussed below in Table 50 of the dissolution rate of a batch of HPMC for all dosage forms and for a single dosage form made from a batch for any hydrophilic swellable matrix tablet. Thus, the ability to achieve a selected dissolution rate for each time point over an extended period of time for a dissolution profile can easily be planned and achieved when designing an extended release tablet having a hydrophilic swellable matrix.

HPMC is a polymer that modulates a drug release rate in a hydrophilic swellable matrix tablet or dosage form. HPMC can be found in tablets containing many different active ingredients (variously, “actives” or APIs), including but not limited to, tetracycline-class compounds such as tetracycline, minocycline and doxycycline, and their pharmaceutically-acceptable salts and hydrates. The drug release rate depends on several formulation variables such as the concentration of the active and water solubility of the active.

Unexpectedly, the present disclosure contemplates that in an extended release tablet having Hypromellose in an amount of from about 20 wt % to 30 wt %, it is possible to vary the % HP to achieve a desired dissolution profile that has at least 80% f2 similarity to the desired dissolution profile and consistent across all the dosage forms. The factor f2 is a measurement of the closeness between a reference and a test profile. In dissolution profile comparisons, especially to assure product performance, regulatory interest is in knowing how similar the profiles are, and to have a measure that is more sensitive to large differences at any particular time point. When the two profiles are identical, f2=100. An average difference of 10% at all measured time points results in a f2 value of 50%. The FDA has set a public standard of f2 value between 50-100 to indicate similarity between two dissolution profiles

The following formula is used to calculate f2, where Rₜ and Tₜ are cumulative percent dissolved at each of n selected time points of a reference and a test formulation respectively:

f₂=50 log {[1+(1/n)Σₜ₌₁n(Rₜ−Tₜ)2]−0.5·100}

Further, the present disclosure contemplates that by varying the % HP, it is possible to either increase or decrease the dissolution rate of the active. It has unexpectedly been discovered that an increase in the % HP will result in an increase in the dissolution rate of the active. Conversely, it has been found that a decrease in the % HP will result in a decreased dissolution rate.

While the extended tablets specifically described as being produced and evaluated in the present disclosure have minocycline HCl as the active, it is believed that tablets having any tetracycline class compounds and even any other water soluble actives will exhibit similar properties in terms of a controlled dissolution profile, ability to control the dissolution profile through varying % HP, etc. It is believed that in another exemplary embodiment, the tablet active would be doxycycline, or a hydrate thereof.

Again, in the following examples, each tablet has the highly-soluble hydrochloride salt of minocycline HCl as the active ingredient (unless otherwise mentioned) references to minocycline herein should be understood as referring to minocycline HCl; although it is contemplated that the principles of the invention can be applied to other forms of minocycline and minocycline derivatives and analogs. It has been unexpectedly found that the percent hydroxypropoxyl substitution of the HPMC plays a critical role in the rate of dissolution of minocycline HCl from the dosage form or tablet for a range of dosage forms, or strengths. One commercially-available HPMC tested, Methocel E50 Premium LV from SUPPLIER 01 typically has a % HP of from about 7% to about 12%.

The amount or concentration of Hypromellose for the 45 mg, 65 mg, 90 and 115 mg dosage forms of minocycline is 27 wt %, and the amount of concentration of Hypromellose is 23.5 wt % for the 135 mg dosage form. As used herein, the term “strength” in connection with a tetracycline-class dosage form means that the dosage form comprises a given free base equivalent amount of the tetracycline-class compound. Thus, 45 mg strength comprises a free base equivalent amount of 45 mg of tetracycline-class compound. The concentration of this rate controlling polymer is based on a core weight of 400 mg for the examples and tests discussed herein. As used herein the term “core weight” as applied to a tablet means a weight of the tablet before the tablet is encapsulated, coated, or otherwise contained.

Cellulose is a polymer comprising many anhydroglucose sugar molecules. Collectively, these sugar monomers when polymerized as cellulose are referred to as a cellulose chain. The term “degree of substitution” is the average level of methoxyl substitution on the cellulose chain. This average value is typically a real number between above 0 and 3, corresponding to three possible sites of substitution on each sugar molecule. However, since hydroxypropoxyl bases can be attached to each other, i.e., independent of a base substitution site on the cellulose molecule, the number can be higher than 3. Degree of substitution is often expressed as “molar substitution”, which is the average level of hydroxypropoxyl substitution on the cellulose chain.

Synthesis of HPMC from a cellulose chain is carried out with propylene oxide and methyl chloride to obtain hydroxypropyl substitution on the anhydroglucose units of the cellulose chain. This substituent group, —OCH2CH(OH)CH3—, contains a second hydroxyl on the number two carbon and may also be considered to form a propylene glycol ether of cellulose. These products possess varying ratios of hydroxypropyl and methyl substitution, a factor that influences organic solubility and the thermal gelation temperature of aqueous solutions.

In the present disclosure, the overall objective of the tablet production was to scale-up the manufacture of a range of oral dosage forms having extended release (ER) that is capable of gradually delivering 90% of minocycline HCl 45, 90 or 135 mg, as free base over a 4 hour period for use in the once-daily treatment of acne vulgaris.

The dosage forms were developed by systematically varying the level of polymer Hypromellose in the dosage form while keeping constant the total tablet weight. The process used to manufacture the dosage form includes high shear granulation followed by wet milling and fluid bed drying. The formulation is then dry milled and blended with extra-granular excipients before being compressed into a tablet or dosage form and subsequently film coated.

Methocel Cellulose Esters (like Hypromellose, USP, type 2910) are the first choice for the formation of hydrophilic matrix systems, providing a robust mechanism for the slow release of actives or drugs from oral solid dosage forms. Methocel provides a simple solution to meet a range of drug solubility needs. Tablets are manufactured with existing, conventional equipment and processing methods. The polymer of choice was Hypromellose, USP; type 2910 (Methocel E50 Premium LV), which provided the desired release profiles in concentrations between 20 and 30 wt %. Again, polymer concentration chosen for the 135 mg dosage form was 23.5 wt %. The polymer concentration for the 45 and 90 mg dosage forms is identical at 27 wt %.

A dissolution criterion of approximately 90% released at 4 hours and dissolution ranges for 1 and 2 hour time points were established for all dosage forms of minocycline tablets produced and studied.

The following experimental data, exemplary HPMC pharmaceutical forms (minocycline HCl tablets), and related disclosure, are intended to further illustrate particular aspects of the present disclosure, without limiting the overall scope of the present disclosure.

Accumulated Stability Data for Registration and Clinical Batches of Minocycline Extended Release Tablets

The 45 mg and 90 mg minocycline dosage forms produced and analyzed in the present disclosure have the same amount or concentration of polymer (HPMC), and same % HP, and therefore should exhibit similar dissolution profiles. Clinical, registration, and validation data support this prediction. The lower amount or concentration of polymer in the 135 mg dosage form displays a slightly higher release at the 2-hour time point.

In each of Tables 1-25, the results of dissolution tests on various batches of a dosage form or tablet having a specified amount of minocycline hydrochloride measured in milligrams (mg) are shown. In each table, each of the various batches (e.g., batch 1, 2, and 3, etc.) have consistent amounts of constituents (except as noted herein) except for the percent hydroxypropoxylation of the HPMC, where indicated in the table. The results from the tables demonstrate that all other factors being consistent, when the % HP decreases, there is a resulting decrease in the mean dissolution rate. Otherwise stated, an increase in the % HP substitution resulted in an increased linear mean dissolution rate.

TABLE 1
135 mg Minocycline HCL Tablets Dissolution Profiles

| | Spec | | | |
|---|---|---|---|---|
| | 35-50% | 67-82% | NLT 90% | |
| Lot | 1 hour | 2 hour | 4 hour | % HP |
| Batch 1 | 43.167 | 69.833 | 101.5 | 8.3 |
| Batch 2 | 41.167 | 69.667 | 99.667 | 8.3 |
| Batch 3 | 41.5 | 71.167 | 100.5 | 8.3 |
| Batch 4 | 41.333 | 70.667 | 101.833 | 8.3 |
| Mean | 41.79175 | 70.3335 | 100.875 | |
| SP | 0.926857549 | 0.707263977 | 0.984594333 | |

NDA

| | | | | |
|---|---|---|---|---|
| NDA 04012 | 42.667 | 72.833 | 102.833 | 8.7 |
| NDA 04011 | 40.667 | 69 | 100.67 | 8.7 |
| NDA 03292 | 42.167 | 72 | 100.17 | 8.4 |
| Mean | 41.83366667 | 71.27766667 | 101.2243333 | |
| SD | 1.040833 | 2.016010003 | 1.415399708 | |

Table 1 shows that (a) the 135 mg dissolution profiles were more consistent than NDA; and (b) the level of % HP, namely, 8.3; 8.4-8.7; 8.7 to 9.1; and 9.1 to 9.8 effect the ability to be more consistent.

TABLE 2
135 mg Minocycline HCL Tablets with 8.3%
HP, 9.8% HP and NDA batches (8.4-8.7% HP)

spec

| | 35-50% | 67-82% | NLT 90% | |
|---|---|---|---|---|
| | 1 hour | 2 hour | 4 hour | % HP |
| Lot Number | | | | |
| 9A5482 | 43.167 | 69.833 | 101.5 | 8.3 |
| 9A5483 | 41.167 | 69.667 | 99.667 | 8.3 |
| 9A5484 | 41.5 | 71.167 | 100.5 | 8.3 |
| 8T5414 | 41.333 | 70.667 | 101.833 | 8.3 |
| 8N4995 | 45 | 80 | 100 | 9.8 |
| 8P5281 | 43 | 77 | 102 | 9.8 |
| 8P5282 | 44 | 78 | 101 | 9.8 |
| NDA 04012 | 42.667 | 72.833 | 102.833 | 8.7 |
| NDA 04011 | 40.667 | 69 | 100.67 | 8.7 |
| NDA 03292 | 42.167 | 72 | 100.17 | 8.4 |
| HPMC % HP | | | | |
| mean 8.3% | 41.79175 | 70.3335 | 100.875 | |
| mean 8.4% | 42.167 | 69 | 100.67 | |
| mean 8.7% | 41.667 | 70.9165 | 101.7515 | |
| mean 9.8% | 44 | 78.33333 | 101 | |

TABLE 3
Minocycline HCL Tablets with 8.3% HP HPMC
45 mg commercial
8.3% HP

spec

| | 35-50% | 60-75% | NLT 90% | |
|---|---|---|---|---|
| Batch | 1 hour | 2 hour | 4 hour | HMPC |
| 9B5650 | 38.5 | 62.1667 | 99 | 8.3% HP |
| 9B5651 | 39.167 | 63.167 | 98.167 | |
| 9B5649 | 37.833 | 61.5 | 97.667 | |
| 9B5648 | 38.167 | 62.833 | 99.667 | |
| 9B5652 | 38.333 | 63.167 | 100.167 | |
| mean | 38.4 | 62.56674 | 98.9336 | |

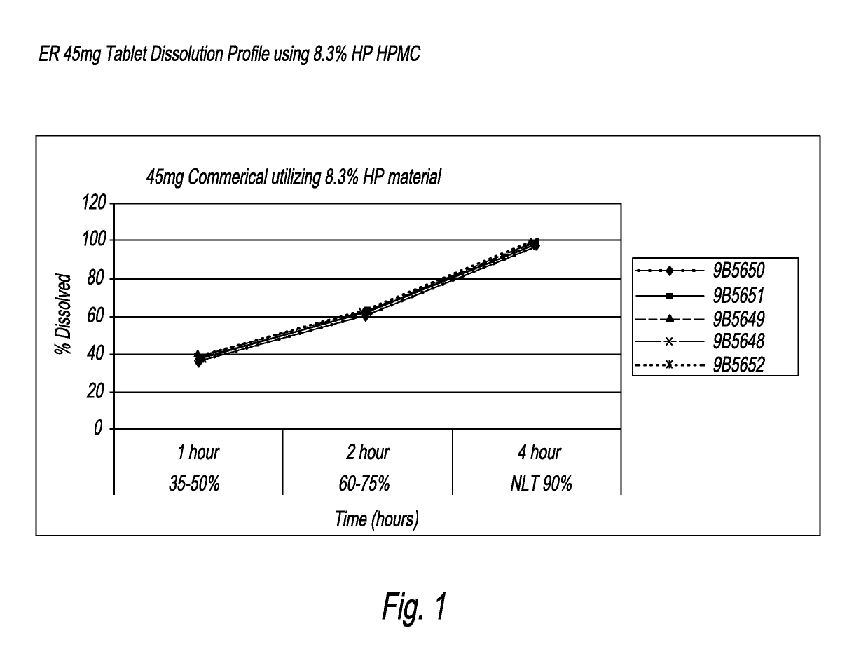

Referring to FIG. 1, the dissolution profile of 45 mg tablets using 8.3% HP HMPC based on the experimental results of Table 3 is shown.

TABLE 4
Comparison of 45 mg Tablets with 8.3%
HP HPMC versus 45 mg NDA batches

spec

| | 35-50% | 60-75% | NLT 90% | |
|---|---|---|---|---|
| Lot Number | 1 hour | 2 hour | 4 hour | % HP |
| NDA 4008 | 39.667 | 64.667 | 102 | 8.4 |
| NDA 4007 | 39.667 | 64.833 | 102 | 8.4 |
| NDA 3290 | 38.167 | 63.1667 | 102 | 8.4 |
| mean | 39.167 | 64.22223 | 102 | |
| 9B5650 | 38.5 | 62.1667 | 99 | 8.3% |
| 9B5651 | 39.167 | 63.167 | 98.167 | |
| 9B5649 | 37.833 | 61.5 | 97.667 | |
| 9B5648 | 38.167 | 62.833 | 99.667 | |
| 9B5652 | 38.333 | 63.167 | 100.167 | |
| mean | 38.4 | 62.6 | 98.9 | |
| NDA mean | 39.2 | 64.2 | 102 | |
| 45 mg 8.3% mean | 38.4 | 62.6 | 98.9 | |

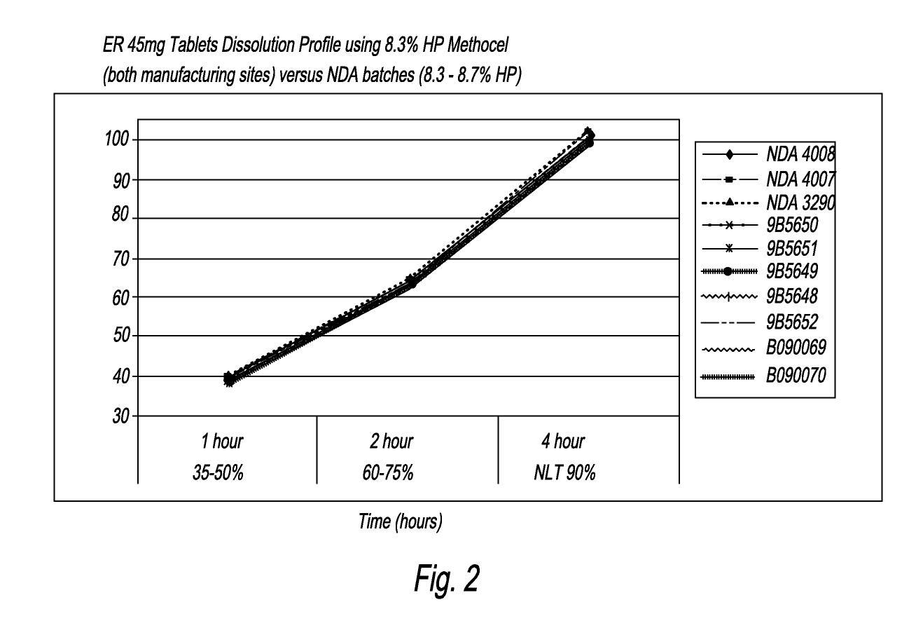

Referring to FIG. 2, the dissolution profile of 45 mg tablets using 8.3% HP HPMC versus NDA batches (8.3-8.7% HP) based on the experimental results of Table 4 is shown.

TABLE 5
45 mg Tablet Dissolution Profile with 8.3% HP HPMC

| | spec | | | |
|---|---|---|---|---|
| | 35-50% | 60-75% | NLT 90% | HMPC |
| Batch | 1 hour | 2 hour | 4 hour | 8.3% HP |
| 9B5650 | 38.5 | 62.1667 | 99 | |
| 9B5651 | 39.167 | 63.167 | 98.167 | |
| 9B5649 | 37.833 | 61.5 | 97.667 | |
| 9B5648 | 38.167 | 62.833 | 99.667 | |
| 9B5652 | 38.333 | 63.167 | 100.167 | |
| mean - | 38.4 | 62.56674 | 90.9336 | |
| TESTER_02 | | | | |
| B090069 | 38.667 | 62.833 | 98.5 | |
| B090070 | 38.833 | 62 | 98 | |
| mean - | 38.3665 | 62.86687 | 99.5503 | |
| TESTER_01 | | | | |

TABLE 6
45 mg ER Tablets Dissolution Profiles 8.3%
HP HPMC vs. NDA batches (8.3-8.7% HP)

spec

| | 35-50% | 60-75% | NLT 90% | |
|---|---|---|---|---|
| Lot Number | 1 hour | 2 hour | 4 hour | % HP |
| NDA 4008 | 39.667 | 64.667 | 102 | 8.4 |
| NDA 4007 | 39.667 | 64.833 | 102 | 8.4 |
| NDA 3290 | 38.167 | 63.1667 | 102 | 8.4 |
| 9B5650 | 38.5 | 62.1667 | 99 | 8.3 |
| 9B5651 | 39.167 | 63.167 | 98.167 | 8.3 |
| 9B5649 | 37.833 | 61.5 | 97.667 | 8.3 |
| 9B5648 | 38.167 | 62.833 | 99.667 | 8.3 |
| 9B5652 | 38.333 | 63.167 | 100.167 | 8.3 |
| B090069 | 38.667 | 62.833 | 98.5 | 8.3 |
| B090070 | 38.833 | 62 | 98 | 8.3 |
| mean | 38.7001 | 63.03334 | 99.7168 | |
| SD | 0.632596 | 1.065176 | 1.7445 | |

TABLE 7
45 mg ER tablets NDA Batches (8.4% HP) Dissolution Profile

| | 35-50% | 60-75% | NLT 90% |
|---|---|---|---|
| Lot Number | 1 hour | 2 hour | 4 hour |
| NDA 4008 | 39.667 | 64.667 | 102 |
| NDA 4007 | 39.667 | 64.833 | 102 |
| NDA 3290 | 38.167 | 63.1667 | 102 |
| mean | 39.167 | 64.22223 | 102 |
| SD | 0.866025 | 0.917879 | 0 |

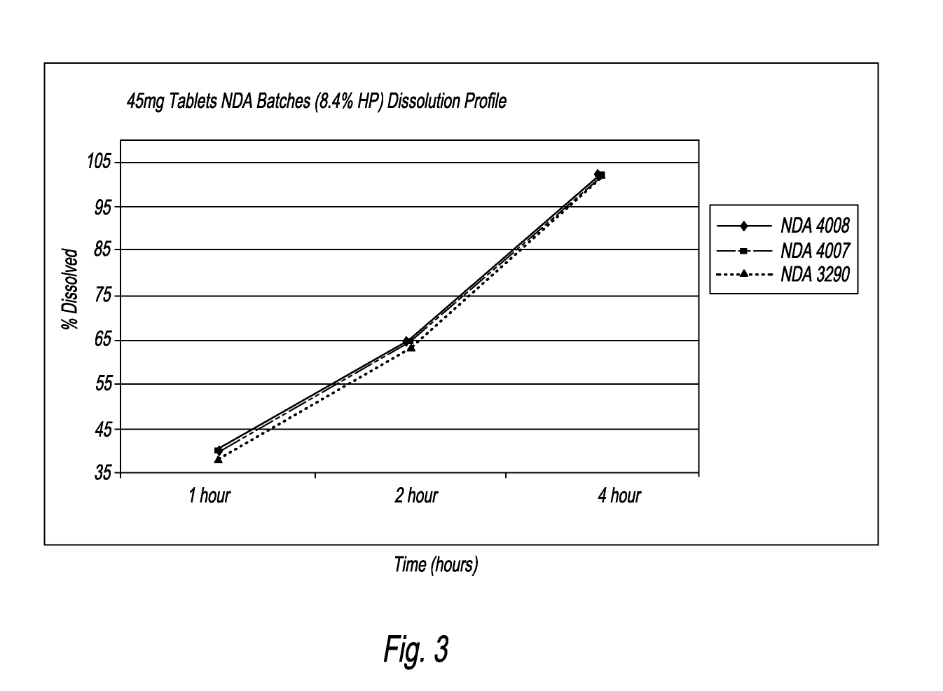

Referring now to FIG. 3, the dissolution profile of 45 mg tablets NDA batches using 8.4% HP HMPC based on the experimental results of Table 7 is shown.

TABLE 8
45 mg ER tablets with 9.4% HP HPMC

spec

| | 35-50% | 60-75% | NLT 90% | |
|---|---|---|---|---|
| Tablet | 1 hour | 2 hour | 4 hour | HMPC |
| B070176 | 43 | 71.67 | 101.5 | 9.4 |
| B070348 | 42 | 70.5 | 104.5 | 9.4 |
| B070178 | 44 | 73.33 | 103 | 9.4 |
| mean | 43 | 71.83333 | 103 | |

TABLE 9
45 mg ER tablets with 8.9% HP Methocel.

spec

| | 35-50% | 60-75% | NLT 90% | |
|---|---|---|---|---|
| Batch | 1 hour | 2 hour | 4 hour | % HP |
| 8H4491 | 40.8 | 70 | 100.2 | 8.9% |
| 8F4284 | 41 | 70.3 | 100.2 | 8.9% |
| 8F4285 | 41.5 | 70.8 | 100.8 | 8.9% |
| 8F4492 | 41.2 | 71 | 100.8 | 8.9% |
| 8F3859 | 41.2 | 69.8 | 100.2 | 8.9% |
| mean | 41.14 | 70.38 | 100.44 | |
| SD | 0.260768 | 0.511859 | 0.328634 | |

TABLE 10
Effect of varying % HP HPMC on 45 mg Tablets Dissolution Profile.
Spec

| 35-50% | 60-75% | NLT 90% | |
|---|---|---|---|
| 1 hour | 2 hour | 4 hour | % HP |
| 38.4 | 62.6 | 98.9 | 8.3 |
| 43 | 71.8 | 103 | 9.4 |
| 41.4 | 70.4 | 100.4 | 8.9 |
| 39.2 | 63.2 | 102 | 8.4 |

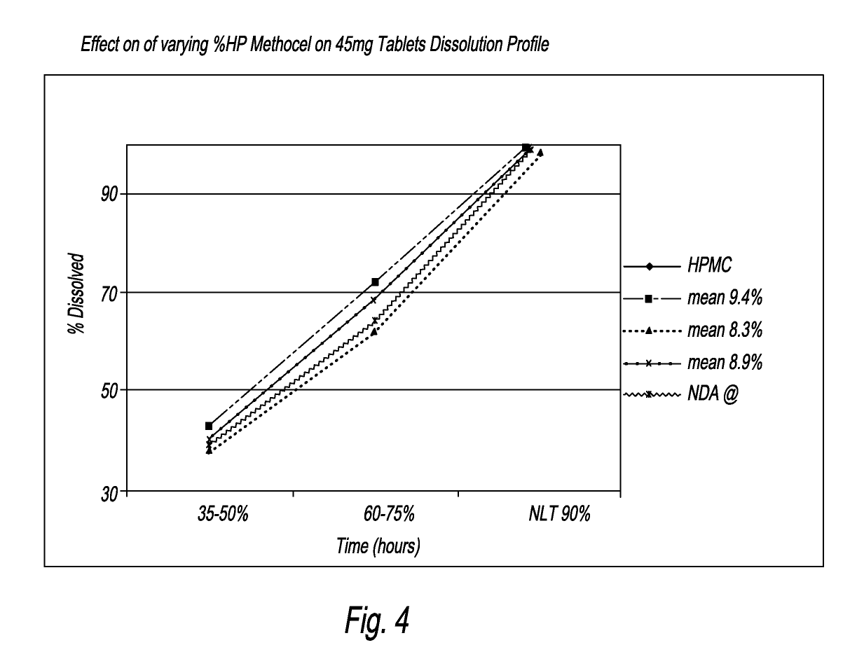

Referring now to FIG. 4, the dissolution profile of 45 mg tablets using 8.3, 9.4, 8.9, and 8.4% HP HMPC based on the experimental results of Table 10 is shown.

TABLE 11
45 mg ER tablet Profile made with 9.8% HP
45 mg with 9.8% HP

| | spec | | | | |
|---|---|---|---|---|---|
| | | 35-50% | 60-75% | NLT 90% | |
| | Tablet | 1 hour | 2 hour | 4 hour | HMPC |
| 8L4953 | 1 | 38 | 65 | 98 | 9.80% |
| | 2 | 41 | 67 | 99 | |
| | 3 | 41 | 69 | 101 | |
| | 4 | 41 | 70 | 101 | |
| | 5 | 41 | 70 | 102 | |
| | 6 | 42 | 70 | 102 | |
| | mean | 40.66667 | 68.5 | 100.5 | |

The Effect of Hydroxypropyl Substitution in HPMC on 90 mg ER Tablets

TABLE 12
90 mg ER tablets dissolution profiles (8.4-8.7% HP vs. 90 mg
ER tablets manufactured with 8.3% HP dissolution profiles)

| NDA | | spec | | |
|---|---|---|---|---|
| Submission | | 35-50% | 60-75% | NLT 90% |
| Batches | Tablet | 1 hour | 2 hour | 4 hour |
| 4009 | 1 | 39 | 63 | 98 |
| % HP 8.4 | 2 | 39 | 63 | 100 |
| | 3 | 37 | 61 | 98 |
| | 4 | 39 | 64 | 98 |
| | 5 | 39 | 63 | 97 |
| | 6 | 39 | 63 | 97 |
| | mean | 38.667 | 62.83 | 98 |
| 4010 | 1 | 39 | 65 | 100 |
| % HP 8.7 | 2 | 39 | 65 | 103 |
| | 3 | 40 | 65 | 103 |
| | 4 | 40 | 65 | 102 |
| | 5 | 41 | 65 | 104 |
| | 6 | 40 | 65 | 101 |
| | mean | 39.833 | 65 | 102.17 |
| 3292 | 1 | 38 | 61 | 94 |
| % HP 8.4 | 2 | 37 | 62 | 96 |
| | 3 | 37 | 62 | 95 |
| | 4 | 38 | 63 | 98 |
| | 5 | 38 | 62 | 98 |
| | 6 | 37 | 62 | 97 |
| | mean | 37.5 | 62 | 96.333 |

| | spec | | | |
|---|---|---|---|---|
| | 35-50% | 60-75% | NLT 90% | 7.0-12.0 |
| Lot Number | 1 hour | 2 hour | 4 hour | % HP |
| 9A5481 | 37.333 | 62.833 | 98.667 | 8.3 |
| 9A5479 | 36.5 | 61.667 | 98.333 | 8.3 |
| 9A5480 | 37.167 | 63.833 | 98.833 | 8.3 |
| NDA 4009 | 38.667 | 62.833 | 98 | 8.4 |
| NDA 4010 | 39.833 | 65 | 102.667 | 8.7 |
| NDA 3292 | 37.5 | 62 | 96.333 | 8.4 |
| mean | 37.83333 | 63.02767 | 98.8055 | |
| SD | 1.20639 | 1.226537 | 2.093631 | |

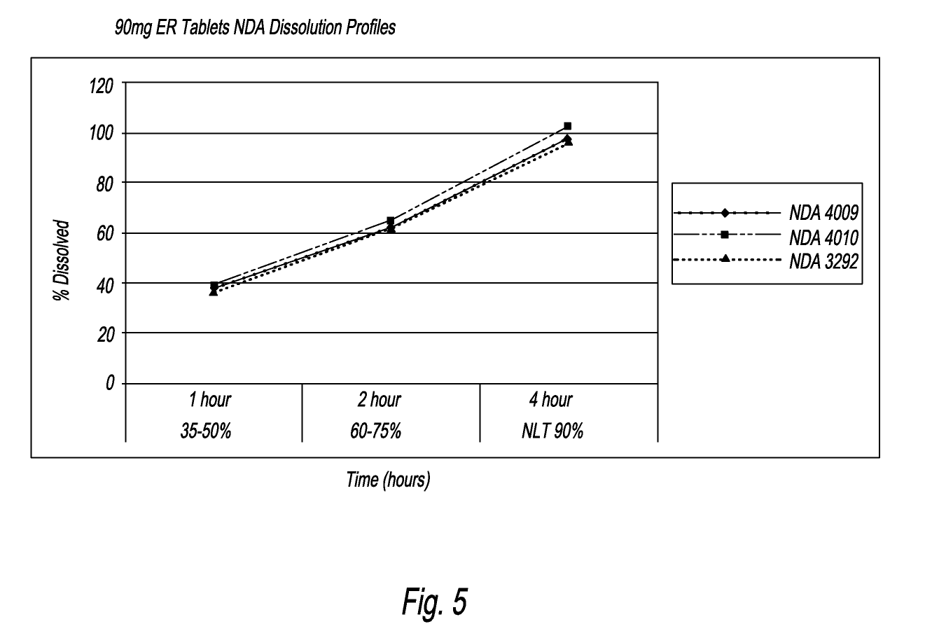

Referring to FIG. 5, the dissolution profile of 90 mg NDA tablets is shown based on the experimental results of Table 12.

TABLE 13
90 mg ER tablets dissolution profile
with 9.8% HP HPMC 90 mg with 9.8% HP

spec

| | 35-50% | 60-75% | NLT 90% | |
|---|---|---|---|---|
| Lot Number | 1 hour | 2 hour | 4 hour | % HP |
| 8L4955 | 41.333 | 70.5 | 101.333 | 9.8 |
| 8K4762 | 41.1667 | 70.1667 | 101.333 | 9.8 |
| 8L4953 | 40.667 | 68.5 | 100.5 | 9.8 |
| 8T5414 | 39.833 | 68.833 | 99.833 | 9.8 |
| B080279 | 42.333 | 71 | 104.667 | 9.8 |
| B080280 | 41.667 | 70.1667 | 103.667 | 9.8 |
| mean | 41.16662 | 69.86107 | 101.8888 | |
| SD | 0.856349 | 0.979964 | 1.879005 | |

TABLE 14
90 mg ER tablets dissolution profile with 9.4% HP HPMC

| | | | | |
|---|---|---|---|---|
| B070182 | 40.5 | 70.33 | 101.5 | 9.4% HP |
| B070180 | 39.33 | 66.67 | 101.33 | |
| B070178 | 41.83 | 71.83 | 101.33 | |
| mean | 40.553333 | 69.61 | 101.3866667 | |

TABLE 15a
90 mg ER tablets dissolution profile with 8.9% HP HPMC

| | spec | | |
|---|---|---|---|
| | 35-50% | 60-75% | NLT 90% |
| Batch | 1 hour | 2 hour | 4 hour |
| 8F4279 | 41.8 | 72 | 101.3 |
| 8F4281 | 42.5 | 73.2 | 101.5 |
| 8F4349 | 42 | 71.7 | 99.5 |
| 8F4283 | 41.8 | 72.2 | 101.8 |
| 8D4492 | 40.8 | 69.2 | 100.2 |
| 8D4194 | 41 | 71 | 100.7 |
| 8F4280 | 42.2 | 72.5 | 101.2 |
| mean | 41.72857 | 71.68571 | 100.8857 |
| SD | 0.61837 | 1.288964 | 0.807111 |

TABLE 15b
90 mg ER tablets dissolution profile with 8.3, 8.4, 8.7, 8.9,
9.4, and 9.8% HP HPMC. Data for 8.9% are mean data from Table 15a.

| | | | NLT |
|---|---|---|---|
| spec | 35-50% | 67-82% | 90% |
| mean % HP | 1 hour | 2 hour | 4 hour |
| 8.3 | 37 | 62.8 | 98.6 |
| 8.4 | 38.1 | 62.4 | 97.2 |
| 8.7 | 39.8 | 65 | 98.6 |
| 8.9 | 41.7 | 71.7 | 100.8 |
| 9.4 | 41.1 | 69.7 | 101.1 |
| 9.8 | 41.1 | 69.9 | 101.9 |

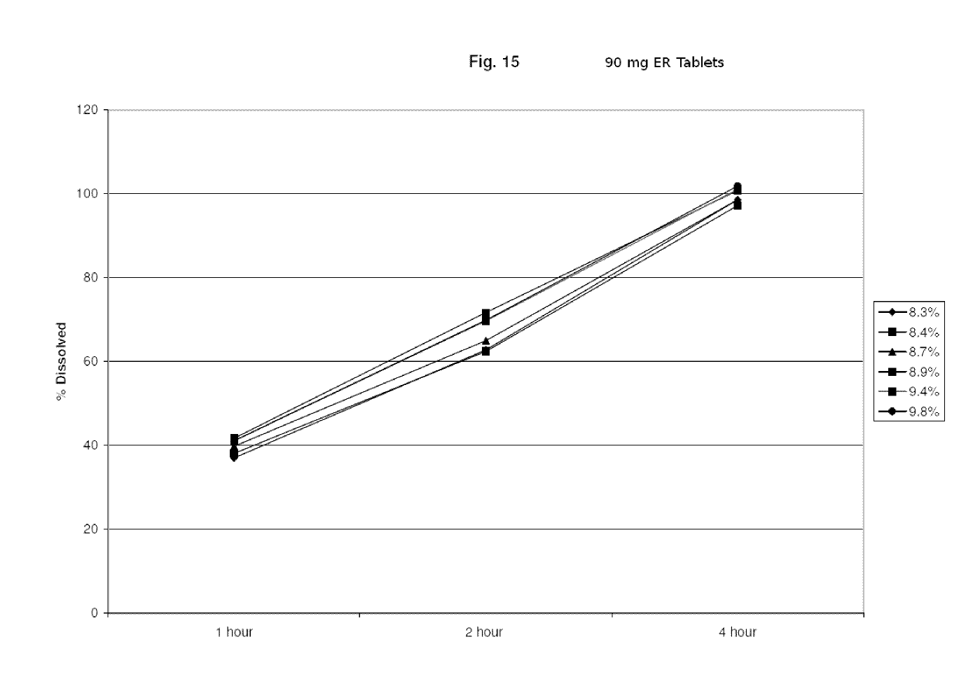

Referring to FIG. 15, there is shown a dissolution profile for a 90 mg ER dosage form according to the experimental Results of Table 15b.

TABLE 16
90 mg ER tablets dissolution profile with 8.8% HP HPMC

| spec | 35-50% | 60-75% | NLT 90% | 7.0-12.0 |
|---|---|---|---|---|
| Lot Number | 1 hour | 2 hour | 4 hour | % HP |
| B060228 | 40.833 | 66.167 | 101.167 | 8.8 |
| B060229 | 39 | 64.667 | 100.67 | 8.8 |
| B060230 | 39.5 | 65.167 | 100.33 | 8.8 |
| mean 90 mg | 39.77767 | 65.33367 | 100.7223 | 8.8 |
| SD | 0.947521 | 0.763763 | 0.420947 | |

TABLE 17
90 mg ER tablets dissolution profile with 8.3% HP HPMC
HPMC −8.3%
HP

| spec | 35-50% | 60-75% | NLT 90% |
|---|---|---|---|
| Lot Number | 1 hour | 2 hour | 4 hour |
| 9C5695 | 36.83 | 61.83 | 96.67 |
| 9C5696 | 38 | 64 | 98.67 |
| 9C5697 | 37.3 | 64 | 98.67 |
| 9C5698 | 37.83 | 61.92 | 97.83 |
| 9C5699 | 36.33 | 60.67 | 98.83 |
| 9C4670 | 37 | 61.83 | 97.67 |
| 9C5761 | 37.29 | 62.12 | 95.95 |
| 9C5762 | 37.61 | 62.87 | 96.71 |
| 9C5763 | 37.23 | 62.41 | 96.67 |
| 9C5764 | 37.32 | 62.78 | 97.95 |
| mean | 37.274 | 62.443 | 97.562 |
| SD | 0.485048 | 1.022981 | 1.011762 |

TABLE 18
Effect of 9.8% HP HPMC on 90 mg Tablets Dissolution
Profile between different Tester sites.

| spec | 35-50% | 60-75% | NLT 90% | % HP |
|---|---|---|---|---|
| Lot Number | 1 hour | 2 hour | 4 hour | 9.8 |
| 8L4955 | 41.333 | 70.5 | 101.333 | |
| 8K4762 | 41.1667 | 70.1667 | 101.333 | |
| 8L4953 | 40.667 | 68.5 | 100.5 | |
| B080279 | 42.333 | 71 | 104.667 | |
| B080280 | 41.667 | 70.1667 | 103.667 | |
| mean | 41.16662 | 69.86107 | 101.8888 | |
| SD | 0.856349 | 0.979964 | 1.879005 | |
| 8L4955 | 41.333 | 70.5 | 101.333 | |
| 8K4762 | 41.1667 | 70.1667 | 101.333 | |
| 8L4953 | 40.667 | 68.5 | 100.5 | |
| mean | 40.74993 | 69.49993 | 100.7498 | |
| SD | 0.673624 | 0.98139 | 0.726445 | |
| B080279 | 42.333 | 71 | 104.667 | |
| B080280 | 41.667 | 70.1667 | 103.667 | |
| mean | 42 | 70.58335 | 104.167 | |
| SD | 0.470933 | 0.589232 | 0.707107 | |

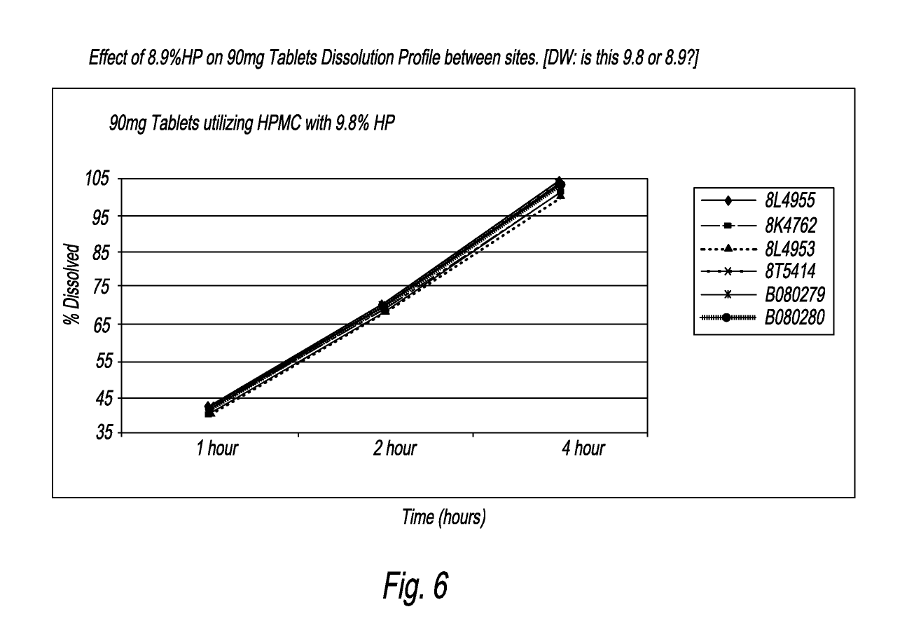

Referring to FIG. 6, the dissolution profile of 90 mg tablets with 9.8% HP HMPC based on the experimental results of Table 18.

TABLE 19
Effect on of varying % HP HPMC on 90 mg tablets
Dissolution Profile

| | | | | |
|---|---|---|---|---|
| spec | 35-50% | 60-75% | NLT 90% | |
| Lot Number | 1 hour | 2 hour | 4 hour | % HP |

90 mg with 9.8% HP

| | | | | |
|---|---|---|---|---|
| 8L4955 | 41.333 | 70.5 | 101.333 | 9.8 |
| 8K4762 | 41.1667 | 70.1667 | 101.333 | 9.8 |
| 8L4953 | 40.667 | 68.5 | 100.5 | 9.8 |
| B080279 | 42.333 | 71 | 104.667 | 9.8 |
| B080280 | 41.667 | 70.1667 | 103.667 | 9.8 |
| mean | 41.16662 | 69.86107 | 101.8888 | |
| SD | 0.856349 | 0.979964 | 1.879005 | |

90 mg at 8.3% HP

| | | | | |
|---|---|---|---|---|
| 9A5481 | 37.333 | 62.833 | 98.667 | 8.3 |
| 9A5479 | 36.5 | 61.667 | 98.333 | 8.3 |
| 9A5480 | 37.167 | 63.833 | 98.833 | 8.3 |
| mean | 37 | 62.77767 | 98.611 | |
| NET EFFECT | −4.17 | −7.08 | −3.06 | |

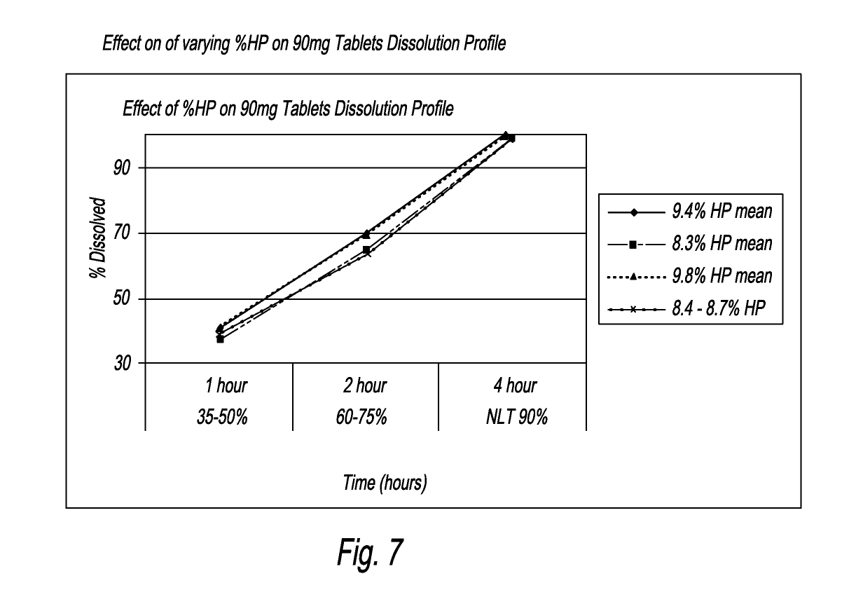

FIG. 7 shows dissolution profiles of 90 mg tablets at varying % HP.

Methocel E50 Premium LV % Hp Substitution Recommendation for 90 mg ER Tablets

Based upon results obtained during NDA, validation and commercial production, the % HP substitution in Methocel E50 Premium LV was determined to fall within the range of 8.3-9.8% HP.

The following data summarizes 135 mg minocycline HCl ER tablets manufactured with HPMC with % HP substitution of 8.3-9.8%.

The 135 mg tablet with its lower HPMC/core tablet weight ratio was the most sensitive to changes in raw materials or process changes. It was first illustrated that the ability to slightly modify the 2 hour time point by varying the coat weight during coating and by increasing the coat weight to 3.9%, the two hour dissolution time point could be decreased by approximately 2%. This was effective in lowering the 2 hour time point from 82% to 80%.

Lot # 8T5414 was manufactured under deviation to adjust a number of critical parameters such as granulation time, mixing times, compression and coating parameters within validated parameters. Dissolution profiles produced were the same as NDA submission batches. Initially, it was thought adjusting these parameters had provided the solution to the dissolution issue. This batch was further reviewed and the next campaign was made only adjusting coating parameters. The resultant dissolution profile of the campaign was the same as 8T5414 and the NDA batches. It was concluded that the process enhancements did not contribute to the change in dissolution profile. An extensive review of all batch documents, raw material certificates of analysis (C of A), Active C of A was performed and it was noted that the % HP substitution of the Methocel E50 Premium LV was 8.3%. From previous investigations, the % HP had been examined but not thought to be underlying factor. Subsequent manufacture utilizing 8.3% HP for 45.90 and 135 mg dosage forms produced dissolution profiles similar (F2>90%) to NDA submission batches.

TABLE 20
135 mg ER tablets NDA Dissolution Profiles

| spec | 35-50% | 67-82% | NLT 90% | |
|---|---|---|---|---|
| Lot Number | 1 hour | 2 hour | 4 hour | % HP |
| NDA 04012 | 42.667 | 72.833 | 102.833 | 8.7 |
| NDA 04011 | 40.667 | 69 | 100.67 | 8.7 |
| NDA 03292 | 42.167 | 72 | 100.17 | 8.4 |
| mean | 41.83367 | 71.27767 | 101.2243 | |
| SD | 1.040833 | 2.01601 | 1.4154 | |

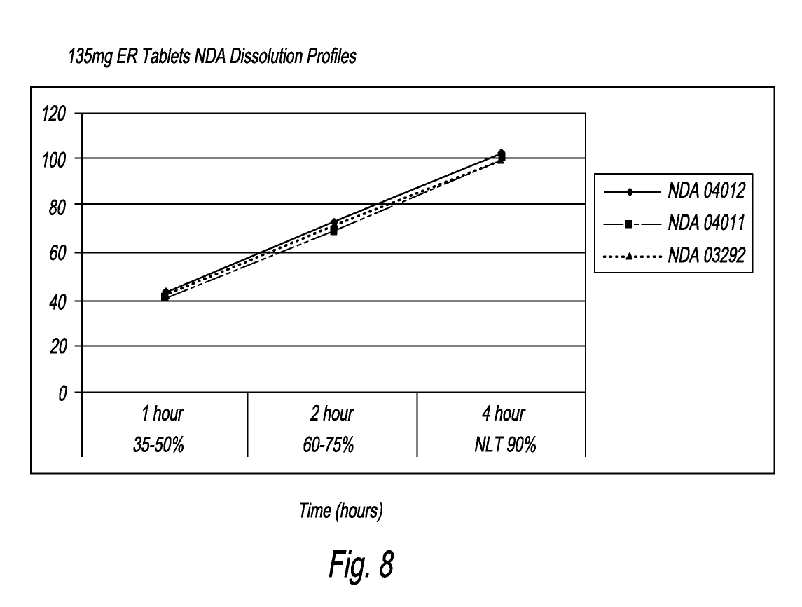

Referring to FIG. 8, the dissolution profile of 135 mg NDA tablets based on the experimental results of Table 20 is shown.

TABLE 21
The effect of varying the % HP of HPMC in 135 mg ER
tablets Dissolution Profile

| spec | 35-50% | 67-82% | NLT 90% | |
|---|---|---|---|---|
| Lot Number | 1 hour | 2 hour | 4 hour | % HP |
| 9A5482 | 43.167 | 69.833 | 101.5 | 8.3 |
| 9A5483 | 41.167 | 69.667 | 99.667 | 8.3 |
| 9A5484 | 41.5 | 71.167 | 100.5 | 8.3 |
| 8T5414 | 41.333 | 70.667 | 101.833 | 8.3 |
| 8N4995 | 45 | 80 | 100 | 9.8 |
| 8P5281 | 43 | 77 | 102 | 9.8 |
| 8P5282 | 44 | 78 | 101 | 9.8 |
| NDA | 42.667 | 72.833 | 102.833 | 8.7 |
| 04012 | | | | |
| NDA | 40.667 | 69 | 100.67 | 8.7 |
| 04011 | | | | |
| NDA | 42.167 | 72 | 100.17 | 8.4 |
| 03292 | | | | |

| HPMC | | | |
|---|---|---|---|
| % HP | 1 hour | 2 hour | 4 hour |
| mean | 41.79175 | 70.3335 | 100.875 |
| 8.3% | | | |
| mean | 42.167 | 69 | 100.67 |
| 8.4% | | | |
| mean | 41.667 | 70.9165 | 101.7515 |
| 8.7% | | | |
| mean | 44 | 78.33333 | 101 |
| 9.8% | | | |

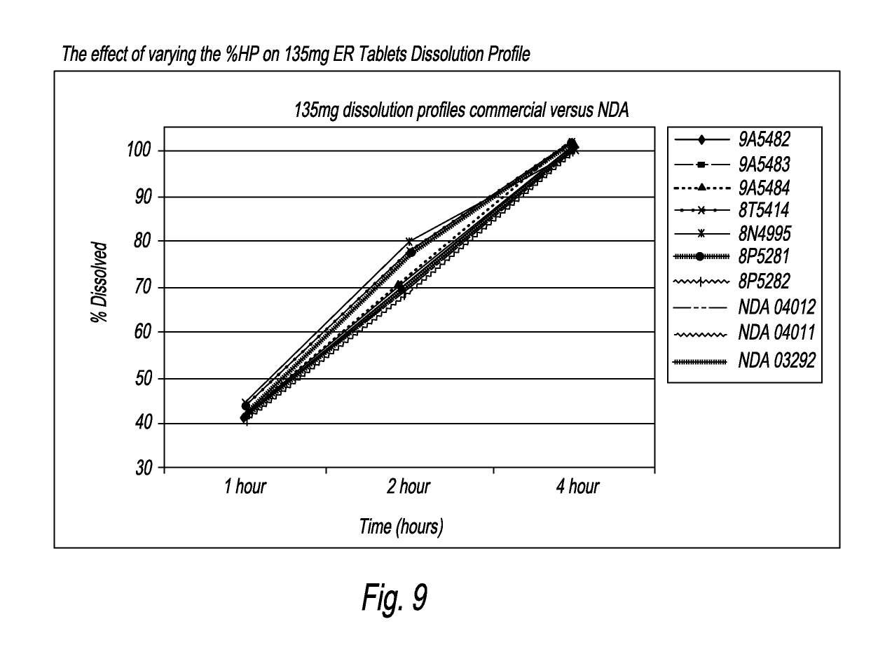

FIG. 9 shows the dissolution profile of 135 mg tablets versus 135 mg NDA tablets based on the experimental results of Table 21.

The following table illustrates the effect of 9.8% HP HPMC on 135 mg ER tablets dissolution profile

TABLE 22
The effect of 9.8% HP Methocel E50 Premium LV on 135 mg
ER tablets dissolution profile
Production with % HP of 9.8

| spec | 35-50% | 67-82% | NLT 90% | HPMC |
|---|---|---|---|---|
| Tablet | 1 hour | 2 hour | 4 hour | 9.80% |
| 8K4763 | 46 | 77 | 100 | |
| 8K4764 | 45 | 79 | 100 | |
| 8K4765 | 45 | 80 | 100 | |
| 8N4955 | 45 | 80 | 100 | |
| mean | 45.25 | 79 | 100 | |

The following Table 23 illustrates the effect of 9.4% HP Methocel E50 Premium LV on 135 mg ER tablets dissolution profile.

TABLE 23
The effect of 9.4% HP HPMC on 135 mg ER tablets dissolution profile
TESTER 01 Production with HPMC % HP of 9.4

| | | spec | | | |
|---|---|---|---|---|---|
| | | 35-50% | 60-75% | NLT 90% | |
| | Tablet | 1 hour | 2 hour | 4 hour | HMPC |
| B070155 | 1 | 47 | 82 | 104 | 9.4% HP |
| | 2 | 45 | 81 | 104 | |
| | 3 | 45 | 80 | 103 | |
| | 4 | 47 | 82 | 102 | |
| | 5 | 46 | 80 | 103 | |
| | 6 | 48 | 83 | 102 | |
| | mean | 46.333333 | 81.33333 | 103 | |
| B070135 | 1 | 45 | 80 | 102 | 9.4% HP |
| | 2 | 46 | 80 | 102 | |
| | 3 | 46 | 81 | 102 | |
| | 4 | 46 | 80 | 102 | |
| | 5 | 47 | 81 | 102 | |
| | 6 | 47 | 79 | 102 | |
| | mean | 46.166667 | 80.16667 | 102 | |
| B070156 | 1 | 48 | 80 | 103 | 9.4% HP |
| | 2 | 46 | 81 | 103 | |
| | 3 | 46 | 81 | 103 | |
| | 4 | 45 | 80 | 103 | |
| | 5 | 45 | 80 | 101 | |
| | 6 | 46 | 82 | 103 | |
| | mean | 46 | 80.66667 | 102.6666667 | |
| | B070155 | 46.33 | 81.33 | 103 | |
| | B070135 | 46.17 | 80.17 | 102 | |
| | B070156 | 46 | 80.67 | 103 | |
| | mean | 46.166667 | 80.72333 | 102.6666667 | |

The following Tables 24 to 27 summarize the effect of % HP substitution (8.3-9.8%) in HPMC on 135 mg ER tablets.

TABLE 24
The effect of % HP substitution (8.3-9.8%) in HPMC on 135 mg
ER tablets.

| spec | 35-50% | 67-82% | NLT 90% | |
|---|---|---|---|---|
| Lot Number | 1 hour | 2 hour | 4 hour | % HP |
| 9A5482 | 43.167 | 69.833 | 101.5 | 8.3 |
| 9A5483 | 41.167 | 69.667 | 99.667 | 8.3 |
| 9A5484 | 41.5 | 71.167 | 100.5 | 8.3 |
| 8T5414 | 41.333 | 70.667 | 101.833 | 8.3 |
| 8N4995 | 45 | 80 | 100 | 9.8 |
| 8P5281 | 43 | 77 | 102 | 9.8 |
| 8P5282 | 44 | 78 | 101 | 9.8 |
| NDA 04012 | 42.667 | 72.833 | 102.833 | 8.7 |
| NDA 04011 | 40.667 | 69 | 100.67 | 8.7 |
| NDA 03292 | 42.167 | 72 | 100.17 | 8.4 |

| HPMC | | | |
|---|---|---|---|
| % HP | 1 hour | 2 hour | 4 hour |
| mean 8.3% | 41.79175 | 70.3335 | 100.875 |
| mean 8.4% | 42.167 | 69 | 100.67 |
| mean 8.7% | 41.667 | 70.9165 | 101.7515 |
| mean 9.8% | 44 | 78.33333 | 101 |

The following Table 25 illustrates the effect of 9.4% HP HPMC on 135 mg ER tablets dissolution profile

TABLE 25
The effect of 9.4% HP Methocel E50 Premium
LV on 135 mg ER tablets dissolution profile.
Production with HPMC % HP of 9.4 for 135 mg

| | | spec | | | |
|---|---|---|---|---|---|
| | | 35-50% | 60-75% | NLT 90% | |
| | Tablet | 1 hour | 2 hour | 4 hour | HMPC |
| B070155 | 1 | 47 | 82 | 104 | 9.4% HP |
| | 2 | 45 | 81 | 104 | |
| | 3 | 45 | 80 | 103 | |
| | 4 | 47 | 82 | 102 | |
| | 5 | 46 | 80 | 103 | |
| | 6 | 48 | 83 | 102 | |
| | mean | 46.333333 | 81.33333 | 103 | |
| B070135 | 1 | 45 | 80 | 102 | 9.4% HP |
| | 2 | 46 | 80 | 102 | |
| | 3 | 46 | 81 | 102 | |
| | 4 | 46 | 80 | 102 | |
| | 5 | 47 | 81 | 102 | |
| | 6 | 47 | 79 | 102 | |
| | mean | 46.166667 | 80.16667 | 102 | |
| B070156 | 1 | 48 | 80 | 103 | 9.4% HP |
| | 2 | 46 | 81 | 103 | |
| | 3 | 46 | 81 | 103 | |
| | 4 | 45 | 80 | 103 | |
| | 5 | 45 | 80 | 101 | |
| | 6 | 46 | 82 | 103 | |
| | mean | 46 | 80.66667 | 102.6666667 | |

TABLE 26
The effect of 8.8% HP HPMC on 135 mg ER tablets
dissolution profile
Production with 8.8% HP

| | spec | 35-50% | 67-82% | NLT 90% |
|---|---|---|---|---|
| | Tablet | 1 hour | 2 hour | 4 hour |
| 135 mg | 1 | 43 | 72 | 102 |
| B060226 | 2 | 43 | 73 | 103 |
| | 3 | 43 | 74 | 104 |
| | 4 | 42 | 70 | 102 |
| | 5 | 43 | 71 | 103 |
| | 6 | 44 | 74 | 104 |
| | mean | 43 | 72.33333 | 103 |
| 135 mg | 1 | 42 | 74 | 103 |
| B060227 | 2 | 43 | 76 | 102 |
| | 3 | 45 | 77 | 104 |
| | 4 | 40 | 72 | 102 |
| | 5 | 42 | 74 | 98 |
| | 6 | 44 | 76 | 102 |
| | mean | 42.66667 | 74.83333 | 101.8333 |
| | B060226 | 43 | 72.333 | 103 |
| | B060227 | 42.667 | 74.833 | 101.83 |
| | mean 135 mg | 42.8335 | 73.583 | 102.415 |

TABLE 27
The effect of % HP substitution HPMC on 135 mg ER tablets

| spec | 35-50% | 67-82% | NLT 90% | |
|---|---|---|---|---|
| Lot Number | 1 hour | 2 hour | 4 hour | % HP |
| 9A5482 | 43.167 | 69.833 | 101.5 | 8.3 |
| 9A5483 | 41.167 | 69.667 | 99.667 | 8.3 |
| 9A5484 | 41.5 | 71.167 | 100.5 | 8.3 |
| 8T5414 | 41.333 | 70.667 | 101.833 | 8.3 |
| B060226 | 43 | 72.333 | 103 | 8.8 |
| B060227 | 42.667 | 74.833 | 101.83 | 8.8 |
| 8N4995 | 45 | 80 | 100 | 9.8 |
| 8P5281 | 43 | 77 | 102 | 9.8 |
| 8P5282 | 44 | 78 | 101 | 9.8 |
| NDA04012 | 42.667 | 72.833 | 102.833 | 8.7 |
| NDA04011 | 40.667 | 69 | 100.67 | 8.7 |
| NDA03292 | 42.167 | 72 | 100.17 | 8.4 |

| HPMC | | | |
|---|---|---|---|
| % HP | 1 hour | 2 hour | 4 hour |
| mean | 41.79175 | 70.3335 | 100.875 |
| 8.3% | | | |
| mean | 42.167 | 69 | 100.67 |
| 8.4% | | | |
| mean | 41.667 | 70.9165 | 101.7515 |
| 8.7% | | | |
| Mean | 42.83 | 73.58 | 102.43 |
| 8.8% | | | |
| mean | 44 | 78.33333 | 101 |
| 9.8% | | | |

dissolution profile.

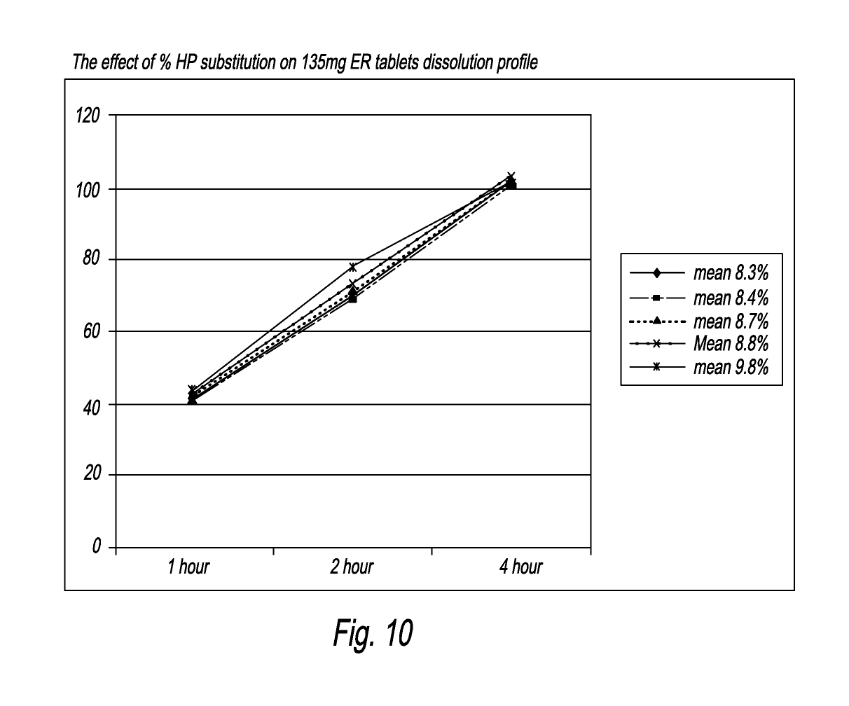

FIG. 10 shows the effect of % HP on 135 mg tablet dissolution profiles, according to the experimental results of Table 27.

65 and 115 Mg Dosage Form ER Tablets:

The ER Tablets with 65 and 115 mg dosage form ER tablets were developed using the same ratio of rate determining polymer (HPMC) as that of the 45 and 90 mg ER tablets, that ratio being 27% polymer ratio to tablet core weight. Since the 115 mg dosage form would have a 27% HPMC/core tablet weight ratio, it was important to ensure a similar dissolution profile to that of all lower dosage forms, or strengths.

TABLE 28
65 mg tablet Validation/Submission Batches Dissolution
Data

| | spec | 35-50% | 60-75% | NLT 90% |
|---|---|---|---|---|
| | Tablet | 1 hour | 2 hour | 4 hour |
| 7K3367 | 1 | 40 | 67 | 99 |
| | 2 | 41 | 67 | 100 |
| | 3 | 41 | 68 | 100 |
| | 4 | 41 | 69 | 101 |
| | 5 | 41 | 69 | 100 |
| | 6 | 41 | 69 | 100 |
| | mean | 40 | 68 | 100 |
| 7K3455 | 1 | 40 | 65 | 100 |
| | 2 | 41 | 66 | 100 |
| | 3 | 42 | 69 | 100 |
| | 4 | 40 | 67 | 102 |
| | 5 | 41 | 67 | 100 |
| | 6 | 42 | 70 | 103 |
| | mean | 41 | 67 | 101 |
| 7L3456 | 1 | 40 | 65 | 101 |
| | 2 | 41 | 68 | 102 |
| | 3 | 40 | 69 | 102 |
| | 4 | 41 | 69 | 103 |
| | 5 | 41 | 69 | 104 |
| | 6 | 42 | 70 | 101 |
| | mean | 41 | 68 | 102 |

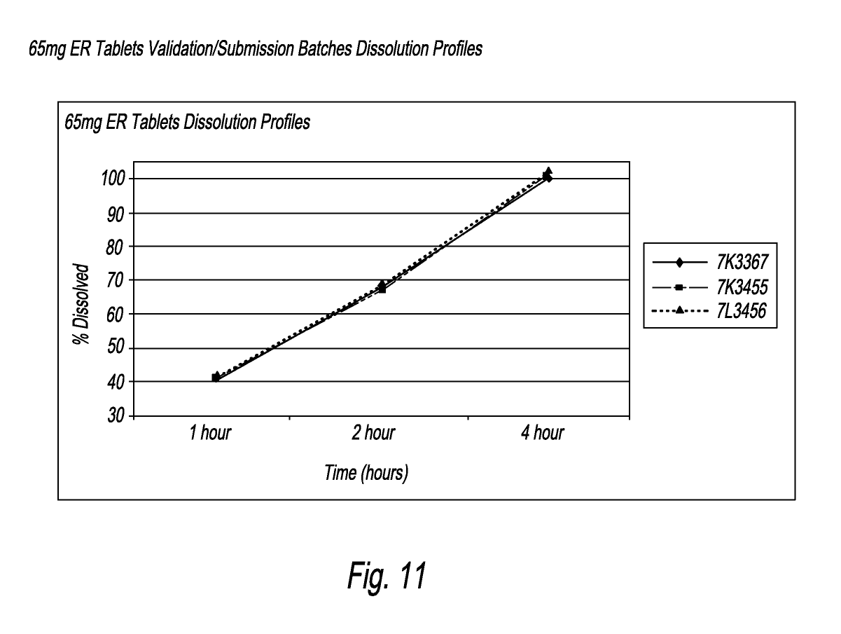

Referring to FIG. 11, the dissolution profile of 65 mg tablets based on the experimental results of Table 28 is shown.

TABLE 29
115 mg Tablet Validation/Submission Batches Dissolution
Data

| | spec | 35-50% | 60-75% | NLT 90% |
|---|---|---|---|---|
| | Tablet | 1 hour | 2 hour | 4 hour |
| 7K3411 | 1 | 38 | 66 | 99 |
| | 2 | 37 | 65 | 102 |
| | 3 | 38 | 67 | 100 |
| | 4 | 38 | 66 | 100 |
| | 5 | 39 | 67 | 100 |
| | 6 | 38 | 66 | 102 |
| | mean | 38 | 66 | 101 |
| 7L3457 | 1 | 39 | 68 | 101 |
| | 2 | 40 | 68 | 100 |
| | 3 | 40 | 68 | 100 |
| | 4 | 39 | 67 | 102 |
| | 5 | 40 | 68 | 102 |
| | 6 | 40 | 68 | 102 |
| | mean | 40 | 68 | 101 |
| 7L3478 | 1 | 38 | 66 | 99 |
| | 2 | 37 | 65 | 102 |
| | 3 | 38 | 67 | 100 |
| | 4 | 38 | 66 | 100 |
| | 5 | 39 | 67 | 100 |
| | 6 | 38 | 66 | 102 |
| | mean | 38 | 66 | 101 |

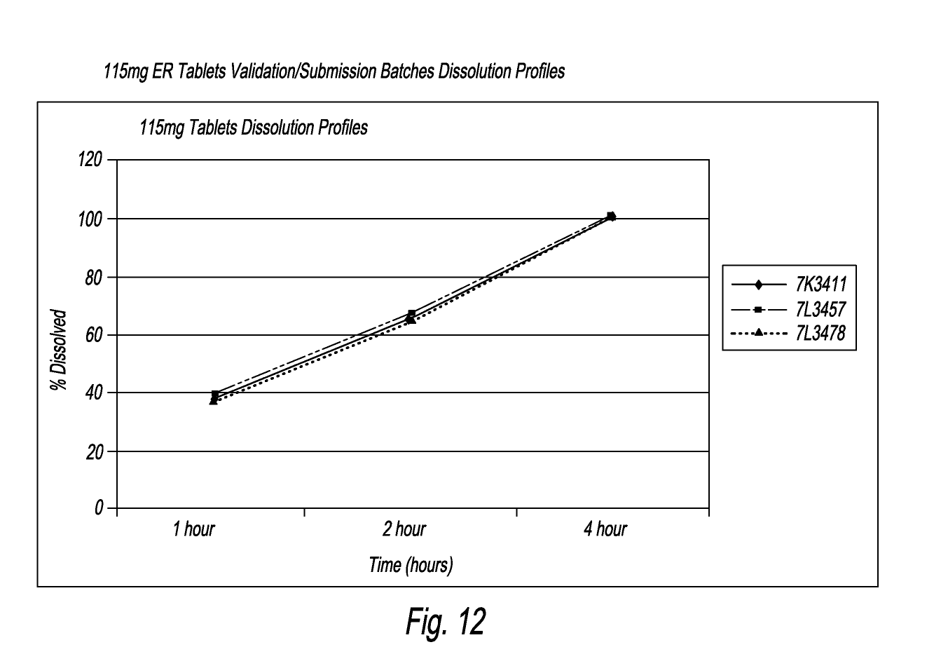

FIG. 12 shows the dissolution profile of 115 mg dosage forms based on the experimental results of Table 29.

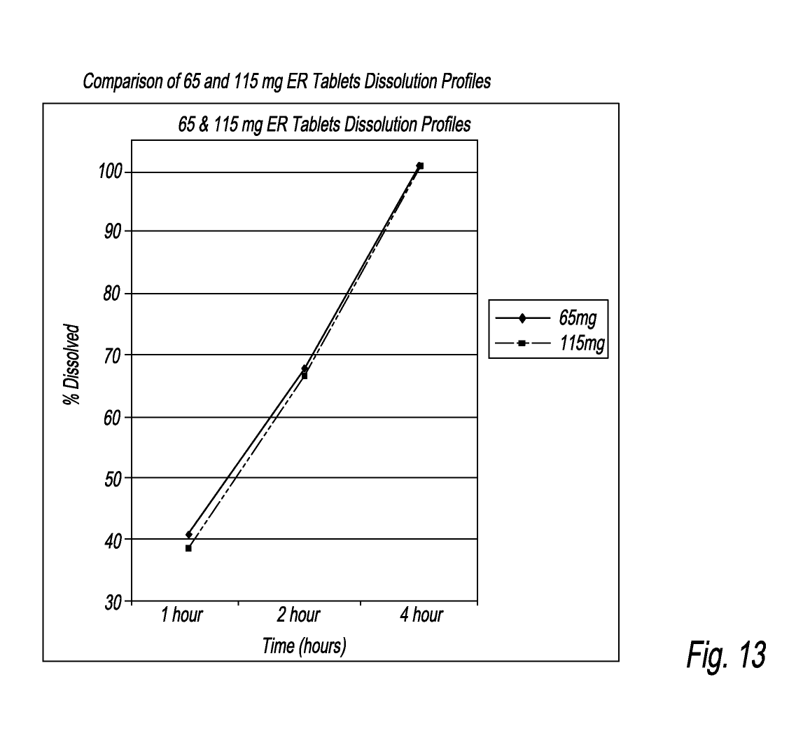

Referring to FIG. 13, there is shown a comparison of the dissolution profile of 65 and 115 mg dosage forms.

65 mg and 115 mg ER tablets or dosage forms submission batches were made with HPMC with a % HP of 8.9-9.1%. 65 mg tablet launch batches were made with 9.8% HP and 8.9% HP. 115 mg tablet launch batches were made with 8.9% HP with one batch containing a split of 8.9 and 9.8% HP. The results of the dissolution testing of the launch batches are summarized below.

TABLE 30
65 mg ER tablets Supplement Batches Dissolution Profile Summary
65 mg

| | | spec | | | |
|---|---|---|---|---|---|
| | | 35-50% | 60-75% | NLT 90% | % HP |
| | Tablet | 1 hour | 2 hour | 4 hour | 7-12% |
| 7K3367 | 1 | 40 | 67 | 99 | TJ09012N23 |
| | 2 | 41 | 67 | 100 | 5121075 |
| | 3 | 41 | 68 | 100 | 9.1 |
| | 4 | 41 | 69 | 101 | |
| | 5 | 41 | 69 | 100 | |
| | 6 | 41 | 69 | 100 | |
| | mean | 41 | 68 | 100 | |
| 7K3455 | 1 | 40 | 65 | 100 | VC24012N21 |
| | 2 | 41 | 66 | 100 | 7090690 |
| | 3 | 42 | 69 | 100 | 9.1 |
| | 4 | 40 | 67 | 102 | |
| | 5 | 41 | 67 | 100 | |
| | 6 | 42 | 70 | 103 | |
| | mean | 41 | 67 | 101 | |
| 7L3456 | 1 | 40 | 65 | 101 | VC24012N21 |
| | 2 | 41 | 68 | 102 | 7090690 |
| | 3 | 40 | 69 | 102 | 9.1 |
| | 4 | 41 | 69 | 103 | |
| | 5 | 41 | 69 | 104 | |
| | 6 | 42 | 70 | 101 | |
| | mean | 41 | 68 | 102 | |

TABLE 31
65 mg ER tablets Launch Batches Dissolution Profile Summary
65 mg Production

| | | spec | | | |
|---|---|---|---|---|---|
| | | 35-50% | 60-75% | NLT 90% | |
| | Tablet | 1 hour | 2 hour | 4 hour | HMPC |
| 8J4511 | 1 | 41 | 69 | 97 | 8060590 |
| | 2 | 40 | 67 | 98 | % HP = 9.8 |
| | 3 | 40 | 68 | 99 | |
| | 4 | 41 | 69 | 104 | |
| | 5 | 41 | 70 | 100 | |
| | 6 | 40 | 68 | 98 | |
| | mean | 41 | 69 | 99 | |
| 8J4506 | 1 | 42 | 72 | 101 | 8060520 |
| | 2 | 42 | 72 | 100 | % HP = 8.9 |
| | 3 | 42 | 72 | 100 | |
| | 4 | 43 | 73 | 102 | |
| | 5 | 42 | 73 | 100 | |
| | 6 | 42 | 72 | 99 | |
| | mean | 42 | 72 | 100 | |
| 8J4507 | 1 | 40 | 66 | 98 | 8060590 |
| | 2 | 41 | 69 | 100 | % HP = 9.8 |
| | 3 | 39 | 68 | 99 | |
| | 4 | 41 | 70 | 99 | |
| | 5 | 41 | 70 | 101 | |
| | 6 | 41 | 69 | 99 | |
| | mean | 41 | 69 | 99 | |
| 8J4512 | 1 | 40 | 69 | 102 | 8060590 |
| | 2 | 41 | 69 | 101 | % HP = 9.8 |
| | 3 | 40 | 69 | 98 | |
| | 4 | 42 | 70 | 102 | |
| | 5 | 41 | 70 | 102 | |
| | 6 | 39 | 67 | 100 | |
| | mean | 41 | 69 | 101 | |
| 8J4510 | 1 | 40 | 69 | 100 | 8060590 |
| | 2 | 41 | 69 | 99 | % HP = 9.8 |
| | 3 | 42 | 70 | 101 | |
| | 4 | 40 | 68 | 99 | |
| | 5 | 40 | 67 | 98 | |
| | 6 | 39 | 67 | 100 | |
| | mean | 40 | 68 | 100 | |
| 8J4509 | 1 | 40 | 66 | 98 | 8060590 |
| | 2 | 41 | 69 | 99 | % HP = 9.8 |
| | 3 | 41 | 70 | 102 | |
| | 4 | 41 | 70 | 102 | |
| | 5 | 41 | 70 | 101 | |
| | 6 | 41 | 71 | 102 | |
| | mean | 41 | 69 | 101 | |
| 8J4508 | 1 | 42 | 71 | 98 | 8060590 |
| | 2 | 42 | 71 | 102 | % HP = 9.8 |
| | 3 | 42 | 70 | 100 | |
| | 4 | 41 | 70 | 101 | |
| | 5 | 41 | 69 | 100 | |
| | 6 | 42 | 69 | 101 | |
| | mean | 42 | 70 | 100 | |

TABLE 32
Summary of 65 mg ER tablets Launch Batches Dissolution
Profiles versus Supplement Batches

| | | | | |
|---|---|---|---|---|
| Spec | 35-50% | 60-75% | NLT 90% | |
| Lot | 1 hour | 2 hour | 4 hour | HPMC % HP |

Launch Batches

| | | | | |
|---|---|---|---|---|
| 8J4511 | 40.5 | 68.5 | 99.3 | 9.8 |
| 8J4506 | 42.2 | 72.3 | 100.3 | 8.9 |
| 8J4507 | 40.5 | 68.7 | 99.3 | 9.8 |
| 8J4512 | 40.5 | 69.0 | 100.8 | 9.8 |
| 8J4510 | 40.3 | 68.3 | 99.5 | 9.8 |
| 8J4509 | 40.8 | 69.3 | 100.7 | 9.8 |
| 8J4508 | 41.7 | 70.0 | 100.3 | 9.8 |
| Mean | 40.9 | 69.5 | 100.0 | |
| SD | 0.706 | 1.390 | 0.644 | |

Supplement Batches

| | | | | |
|---|---|---|---|---|
| 7K3367 | 40.8 | 68.2 | 100.0 | 9.1 |
| 7K3455 | 41.0 | 67.3 | 100.8 | 9.1 |
| 7L3456 | 40.8 | 68.3 | 102.2 | 9.1 |
| Mean | 40.9 | 67.9 | 101.0 | |
| SD | 0.096 | 0.536 | 1.093 | |

In order to determine whether the 65 mg ER tablet made with 9.8% HP behaves the same as the 90 mg ER tablet made with the identical HPMC, the following data are presented.

TABLE 33
90 mg ER tablets Dissolution Profiles made with 9.8% H.P.
HPMC.
90 mg Commercial-9.8% HP

| spec | 35-50% | 60-75% | NLT 90% | 7.0-12.0 |
|---|---|---|---|---|
| Lot Number | 1 hour | 2 hour | 4 hour | % HP |
| 8L4955 | 41.3 | 70.5 | 101.3 | 9.8 |
| 8K4762 | 41.2 | 70.2 | 101.3 | 9.8 |
| 8L4953 | 40.7 | 68.5 | 100.5 | 9.8 |
| 8T5414 | 39.8 | 68.8 | 99.8 | 9.8 |
| B080279 | 42.3 | 71.0 | 104.7 | 9.8 |
| B080280 | 41.7 | 70.2 | 103.7 | 9.8 |
| mean | 41.2 | 69.9 | 101.9 | |
| SD | 0.856 | 0.980 | 1.879 | |

As can be seen from the above tables, the 90 mg and 65 mg ER tablet dissolution characteristics similarly compare with results of 40.9, 69.5% for the 1 and 2 hour time points for the 65 mg and 41.2 and 69.9% for the 90 mg well within the specification of 35-50% for the 1 hour and 60-75% for the two dissolution time points. From this analysis, it is concluded that the 65 mg and 90 mg tablets will have similar dissolution profiles when manufactured with HPMC and the 65 mg tablet dissolution profile behave similarly to the 90 mg tablet dissolution profile regardless of the % HP substitution of the HPMC. Thus, it is possible to use dissolution results (mean of all batches) from 90 mg ER tablets manufactured with HPMC with a % HP substitution of 8.3% to predict the effect on the 65 mg ER tablet if it is manufactured using HPMC having a % HP of 8.3%.

The following Table 34 summarizes the predicted 65 mg ER tablets dissolution profiles made with HPMC with a % HP substitution of 8.3%.

TABLE 34
Predicted effect of 8.3% HP HPMC on 65 mg ER tablets

| | Spec | | |
|---|---|---|---|
| | 35-50% | 60-75% | NLT 90% |
| Strength | 1 hour | 2 hour | 4 hour |
| 65 Predicted | 36.7 | 61.6 | 100.0 |
| 90 Actual | 37.0 | 62.8 | 98.6 |

Table 34 shows predicted 65 mg dissolution profiles.

TABLE 35
Data Used to Calculate Net Effect
of 8.3% HPMC on 65 mg ER tablets

spec

| | | | | |
|---|---|---|---|---|
| | 35-50% | 60-75% | NLT 90% | |
| Lot Number | 1 hour | 2 hour | 4 hour | % HP |

90 mg with 9.8% HP

| | | | | |
|---|---|---|---|---|
| 8L4955 | 41.3 | 70.5 | 101.3 | 9.8 |
| 8K4762 | 41.2 | 70.2 | 101.3 | 9.8 |
| 8L4953 | 40.2 | 68.5 | 100.5 | 9.8 |
| 8T5414 | 39.8 | 68.8 | 99.8 | 9.8 |
| B080279 | 42.3 | 71.0 | 104.7 | 9.8 |
| B080280 | 41.7 | 70.2 | 103.7 | 9.8 |
| Mean | 41.2 | 69.9 | 101.9 | |
| SD | 0.856 | 0.980 | 1.879 | |

90 mg at 8.3% HP

| | | | | |
|---|---|---|---|---|
| 9A5481 | 37.3 | 62.8 | 98.7 | 8.3 |
| 9A5479 | 36.5 | 61.7 | 98.3 | 8.3 |
| 9A5480 | 37.2 | 63.8 | 98.8 | 8.3 |
| Mean | 37.0 | 62.8 | 98.6 | |
| NET EFFECT | −4.17 | −7.08 | −3.06 | |

115 mg ER Tablets Summary and Discussion:

TABLE 36
115 mg ER tablets Supplement Batches Dissolution Profile Summary
115 mg

| | | spec | | | |
|---|---|---|---|---|---|
| | | 35-50% | 60-75% | NLT 90% | % HP |
| | Tablet | 1 hour | 2 hour | 4 hour | 7-12% |
| 7K3411 | 1 | 38 | 66 | 99 | TJ09012N23 |
| | 2 | 37 | 65 | 102 | 5121075 |
| | 3 | 38 | 67 | 100 | 9.1 |
| | 4 | 38 | 66 | 100 | |
| | 5 | 39 | 67 | 100 | |
| | 6 | 38 | 66 | 102 | |
| | mean | 38 | 66 | 101 | |
| 7L3457 | 1 | 39 | 68 | 101 | TJ09012N23 |
| | 2 | 40 | 68 | 100 | 7090690 |
| | 3 | 40 | 68 | 100 | 9.1 |
| | 4 | 39 | 67 | 102 | |
| | 5 | 40 | 68 | 102 | |
| | 6 | 40 | 68 | 102 | |
| | mean | 40 | 68 | 101 | |
| 7L3478 | 1 | 38 | 66 | 99 | TJ09012N23 |
| | 2 | 37 | 65 | 102 | 7090690 |
| | 3 | 38 | 67 | 100 | 9.1 |
| | 4 | 38 | 66 | 100 | |
| | 5 | 39 | 67 | 100 | |
| | 6 | 38 | 66 | 102 | |
| | mean | 38 | 66 | 101 | |

spec

| | | 35-50% | 60-75% | NLT 90% | |
|---|---|---|---|---|---|
| | Lot | 1 hour | 2 hour | 4 hour | |
| means | 7K3411 | 38.0 | 66.2 | 100.5 | 9.1 |
| | 7L3457 | 39.7 | 67.8 | 101.2 | 9.1 |
| | 7L3478 | 38.0 | 66.2 | 100.5 | 9.1 |
| | mean | 38.6 | 66.7 | 100.7 | |
| | SD | 0.962 | 0.962 | 0.385 | |

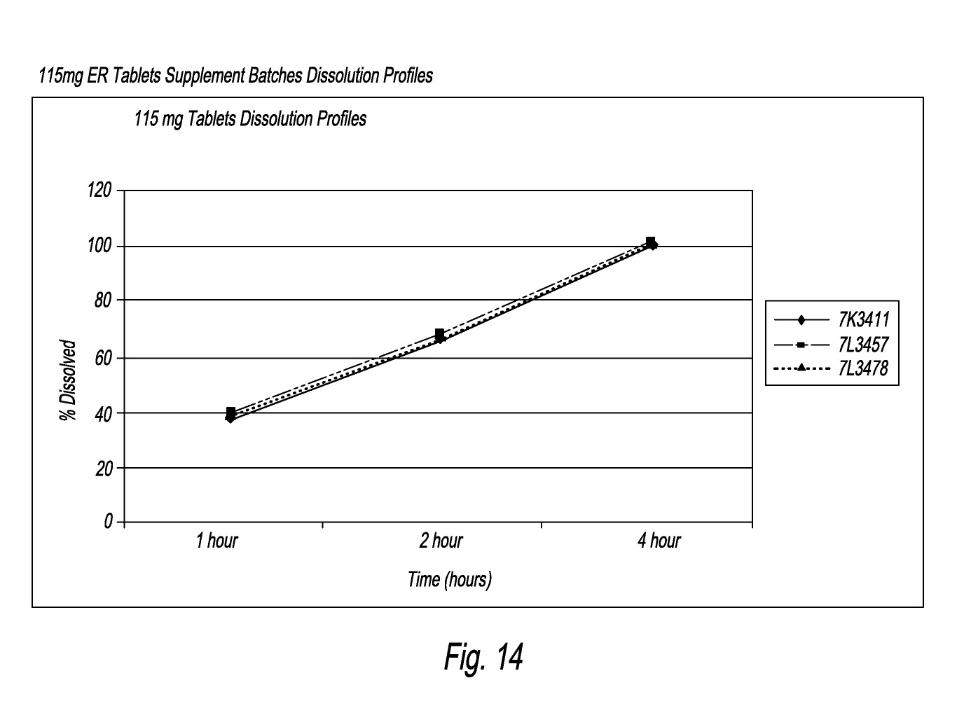

Referring to FIG. 14, the dissolution profile of 115 mg ER tablets based on the experimental results of Table 36 is shown.

TABLE 37
115 mg ER tablets Launch Batches Dissolution Profile Summary
115 mg

| | | spec | | | |
|---|---|---|---|---|---|
| | | 35-50% | 60-75% | NLT 90% | |
| | Tablet | 1 hour | 2 hour | 4 hour | HMPC |
| 8J4549 | 1 | 38 | 67 | 98 | 8060590 |
| | 2 | 37 | 67 | 101 | % HP = 9.8 |
| | 3 | 38 | 68 | 101 | 8060520 |
| | 4 | 39 | 70 | 101 | % HP = 8.9 |
| | 5 | 39 | 69 | 100 | |
| | 6 | 40 | 70 | 100 | |
| | Mean | 39 | 69 | 100 | |
| 8J4548 | 1 | 41 | 70 | 100 | 8060520 |
| | 2 | 40 | 71 | 99 | % HP = 8.9 |
| | 3 | 40 | 71 | 101 | |
| | 4 | 40 | 71 | 99 | |
| | 5 | 41 | 71 | 98 | |
| | 6 | 39 | 69 | 100 | |
| | mean | 40 | 71 | 100 | |
| 8J4547 | 1 | 41 | 69 | 102 | 8060520 |
| | 2 | 40 | 70 | 101 | % HP = 8.9 |
| | 3 | 41 | 72 | 100 | |
| | 4 | 41 | 72 | 101 | |
| | 5 | 40 | 70 | 101 | |
| | 6 | 40 | 72 | 101 | |
| | mean | 41 | 71 | 101 | |
| 8J4546 | 1 | 42 | 71 | 100 | 8060520 |
| | 2 | 40 | 70 | 100 | % HP = 8.9 |
| | 3 | 40 | 72 | 100 | |
| | 4 | 41 | 71 | 101 | |
| | 5 | 41 | 73 | 101 | |
| | 6 | 41 | 72 | 101 | |
| | mean | 41 | 72 | 101 | |
| 8J4545 | 1 | 40 | 71 | 101 | 8060520 |
| | 2 | 40 | 69 | 98 | % HP = 8.9 |
| | 3 | 39 | 70 | 97 | |
| | 4 | 39 | 70 | 98 | |
| | 5 | 40 | 71 | 100 | |
| | 6 | 40 | 71 | 98 | |
| | mean | 40 | 70 | 99 | |

The following shows the predicted effect of using Methocel E50 Premium LV with a % HP substitution of 8.3%.

TABLE 38
Predicted effect of 8.3% HP HPMC on 65 mg ER tablets

| | Spec | | | |
|---|---|---|---|---|
| | 35-50% | 60-75% | NLT 90% | |
| mean % HP | 1 hour | 2 hour | 4 hour | Strength |
| mean 9.8% | 41.167 | 69.861 | 101.889 | 90 mg |
| mean 9.8% | 40.929 | 69.452 | 100.047 | 65 mg |
| intra % HP | positive 0.238 | positive 0.409 | positive 1.842 | |
| difference | | | | |
| spec | 35-50% | 60-75% | NLT 90% | |
| mean % HP | 1 hour | 2 hour | 4 hour | |
| mean 9.8% | 41.167 | 69.861 | 101.889 | |
| mean 8.3% | 37 | 62.78 | 98.611 | |
| net dif- | 4.167 | 7.081 | 3.278 | |
| ference | | | | |
| intra % HP | positive 0.238 | positive 0.409 | positive 1.842 | |
| difference | | | | |
| over effect | negative 3.929 | negative 6.678 | negative 1.436 | |
| Predicted | 37 | 62.774 | 98.611 | |
| 65 mg using | | | | |
| 8.3% HPMC | | | | |

TABLE 39
Predicted effect of 8.3% HP HPMC on 115 mg ER tablets
NET EFFECT ON the 115 mg tablet using HPMC with a 8.3% HP

| | spec | | |
|---|---|---|---|
| | 35-50% | 60-75% | NLT 90% |
| Lot Number | 1 hour | 2 hour | 4 hour |
| Launch Batches | 39.93 | 70.33 | 99.97 |
| NDA Batches | 38.56 | 66.72 | 100.72 |
| mean | 39.245 | 68.525 | 100.345 |
| Over all effect | −4.167 | −7.08 | −3.056 |
| Predicted 115 mg using | 35.08 | 61.445 | 100* |
| 8.3% HPMC | | | |

The data set forth above evidences a strong correlation between the Hydroxypropyl substitution in the HPMC and the minocycline HCl ER tablet dissolution profile. The assay specification for the % HP substitution in this HPMC is 7.0-12.0%. The ER tablets have been manufactured with HPMC with % HP from 8.3-9.8%. The lower the % HP Methocel used, the greater the percent dissolved at the 1 and 2 hour time point for all dosage forms.

ER tablets 45, 90 and 135 mg were originally developed in the pre-clinical stage with Methocel containing a % HP substitution of 8.4-8.8%. For the submission-biobatches (NDA batches), Methocel containing % HP substitution of 8.4-8.7% was used. 1, 2 and 4 hour dissolution profiles or specifications were based on dissolution profiles produced by these batches. These specifications were 35-50% at 1 hour, 60-75% at 2 hours, and NLT 90% at 4 hours for all strengths.

During validation activities for the 45, 90 and 135 mg batches, 2 hour dissolution results for the 135 mg tablet were found to be at the upper limit of 75%, with failing results at stage I testing. All stage II testing passed with 2 hour dissolution results of less than 75%. A new specification of 67-82% for only the 135 mg tablet was put in place at this time. What was not apparent or appreciated was the effect of the % HP substitution on the dissolution profile of ER tablets. Methocel E 50 Premium LV with a % HP of 8.9-9.2 was used at the time of validation, whereas 8.4-8.7% material was used for submission and development batches. With the 135 mg dosage form having a lower percentage HPMC/core tablet weight and a higher active concentration (highly soluble as well), the effect of the % HP substitution had a greater effect on dissolution. Thus, it was first appreciated that the ratio of active to HPMC is a factor, but not as dominant as % HP.

During manufacture, Methocel E50 Premium LV with % HP of 9.3-9.8% was used for production. Dissolution results at 2 hours for the 135 mg tablet were consistently 80-82%, with many stage I dissolution failures and an FDA alert notice generated on a stability failure for a batch failing at the 2 hour time point with results greater than 82%.

Using Methocel E50 Premium LV with a % HP of 8.3% led to the appreciation that % HP had a direct effect on the dissolution profile of ER tablets. Use of this material resulted in dissolution profiles with matched the profiles of the NDA batches with an F2 similarity factor of >90%.

By reviewing the above data and the net predicted effect on the 65 mg and 115 mg dosage forms, it is hypothesized, with a level of confidence, that the effect of a lower % HP substitution on these strengths and subsequent strengths.

As illustrated in the Tables and Figures referenced above, the present disclosure contemplates that the net effect of utilizing 8.3% HPMC is the following:

For 65 mg:

1 hour time point: 3.93% decrease from supplement batches.

2 hour time point: 6.68% decrease from supplement batches

For 115 mg:

1 hour time point: 4.17% decrease from supplement batches

2 hour time point: 7.08% decrease from supplement batches

Tables 40-48 summarize f2 similarity data that compare dissolution profiles of various NDA, commercial, and experimental dosage forms.

Tables 40-48 show the actual and predicted f2 similarity for each strength manufactured with either 8.3 or 9.8% HP. Utilizing 8.3% HP, it is possible to achieve an f2 similarity of >80% when compared to the same strength or closest strength (i.e. compare 115 mg to 90 mg versus 115 mg to 45 mg). Supplement refers to the 65/115 filing versus the 45, 90 and 135 mg NDA submission.

TABLE 40
45 mg versus 45 mg Summary

| Comparison | f2 Similarity |
|---|---|
| 8.3% vs. 9.8% | 59% |
| 8.3% vs. NDA (8.4%) | 82% |
| 9.8% vs. NDA (8.4%) | 65% |

TABLE 41
90 mg versus 90 mg Summary

| Comparison | f2 Similarity |
|---|---|
| 8.3% vs. 9.8% | 64% |
| 8.3% vs. NDA (8.4%) | 99% |
| 9.8% vs. NDA (8.4%) | 72% |

TABLE 42
135 mg versus 135 mg Summary

| Comparison | f2 Similarity |
|---|---|
| 8.3% vs. 9.8% | 66% |
| 8.3% vs. NDA (8.4%) | 97% |
| 9.8% vs. NDA (8.4%) | 68% |

ER 65 Mg Versus 45 Mg, 90 Mg, and 135 Mg Summary

TABLE 43
ER 65 mg versus 45 mg

| Comparison | f2 Similarity |
|---|---|
| 65 mg supplement vs. 45 mg NDA mean | 79% |
| 65 mg supplement vs. 45 mg 8.3% | 71% |
| predicted 65 mg 8.3% versus 45 mg NDA mean | 87% |

TABLE 44
ER 65 mg versus 90 mg

| Comparison | f2 Similarity |
|---|---|
| 65 mg supplement vs. 90 mg NDA mean | 71% |
| 65 mg supplement vs. 90 mg 8.3% | 69% |
| predicted 65 mg 8.3% versus 90 mg NDA mean | 92% |

TABLE 45
ER 65 mg versus 135 mg

| Comparison | f2 Similarity |
|---|---|
| 65 mg supplement vs. 135 mg NDA mean | 82% |
| 65 mg supplement vs. 135 mg 8.3% | 88% |

ER 115 Mg Versus 45 Mg, 90 Mg, and 135 Mg Summary

TABLE 46
ER 115 mg versus 45 mg

| Comparison | F2 Similarity |
|---|---|
| 115 mg supplement vs. 45 mg NDA mean | 85% |
| 115 mg supplement vs. 45 mg 8.3% | 78% |
| predicted 115 mg 8.3% versus 45 mg NDA mean | 74% |

ER 115 Mg Versus 45 Mg and 90 Mg Summary

TABLE 47
ER 115 mg versus 90 mg

| Comparison | F2 Similarity |
|---|---|
| 115 mg supplement vs. 90 mg NDA mean | 78% |
| 115 mg supplement vs. 90 mg 8.3% | 77% |
| predicted 115 mg 8.3% versus 90 mg NDA mean | 81% |

TABLE 48
ER 115 mg versus 135 mg

| Comparison | F2 Similarity |
|---|---|
| 115 mg supplement vs. 135 mg NDA mean | 73% |
| 115 mg supplement vs. 135 mg 8.3% | 76% |

Table 49 summarizes f2 similarity data for NDA reference 45, 90, and 135 mg tablets. HPMC is present in the 45 and 90 mg tablets of Table 49 at 27 wt %.

| % HP | f2 Similarity |
|---|---|
| 8.3 | <85 |
| 8.4-8.7 | <90 |
| 8.7-9.1 | 69 or greater |
| 9.1-9.8 | 59 or greater |

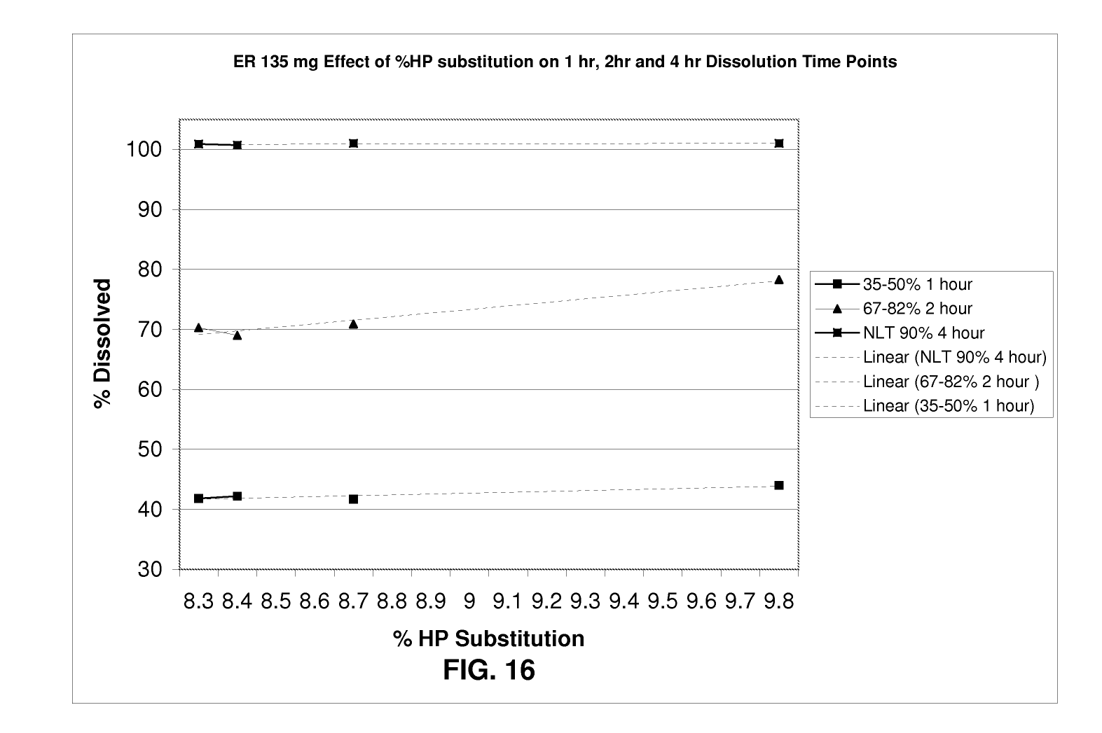

FIG. 16 is a comparison of dissolution profiles of 135 mg ER tablets according to the experimental data that are shown in Table 2. Trend lines for 1-hour timepoints, 2-hour timepoints, and 4-hour timepoints show that percent dissolution at a given timepoint increases as a function of % HP substitution.

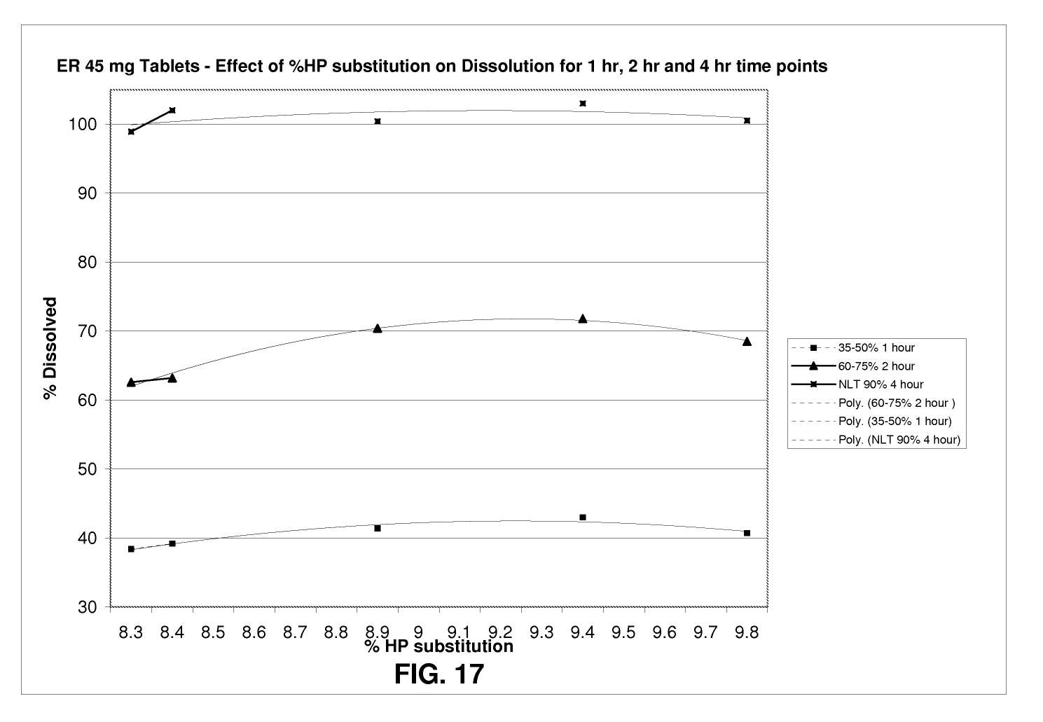

FIG. 17 is a comparison of dissolution profiles of 45 mg ER tablets according to the experimental data that are shown in Tables 10 and 11. Table 11 contains data for 9.8% HP material, and Table 10 contains data for 8.3, 8.4, 8.9, and 9.4% material. Best-fit trend lines for 1-hour timepoints, 2-hour timepoints, and 4-hour timepoints are polynomial, showing that percent dissolution at a given timepoint increases nonlinearly.

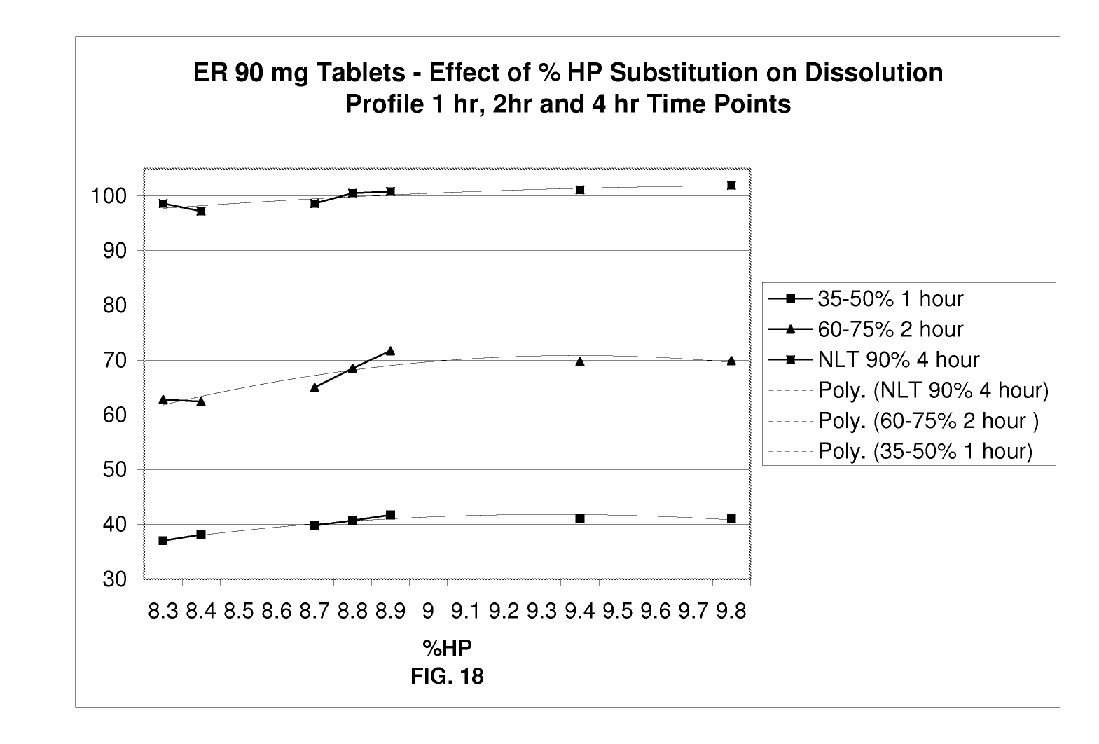

FIG. 18 is a comparison of dissolution profiles of 90 mg ER tablets according to the experimental data that are shown in Table 15a. Best-fit trend lines for 1-hour timepoints, 2-hour timepoints, and 4-hour timepoints are polynomial, showing that percent dissolution at a given timepoint increases nonlinearly.

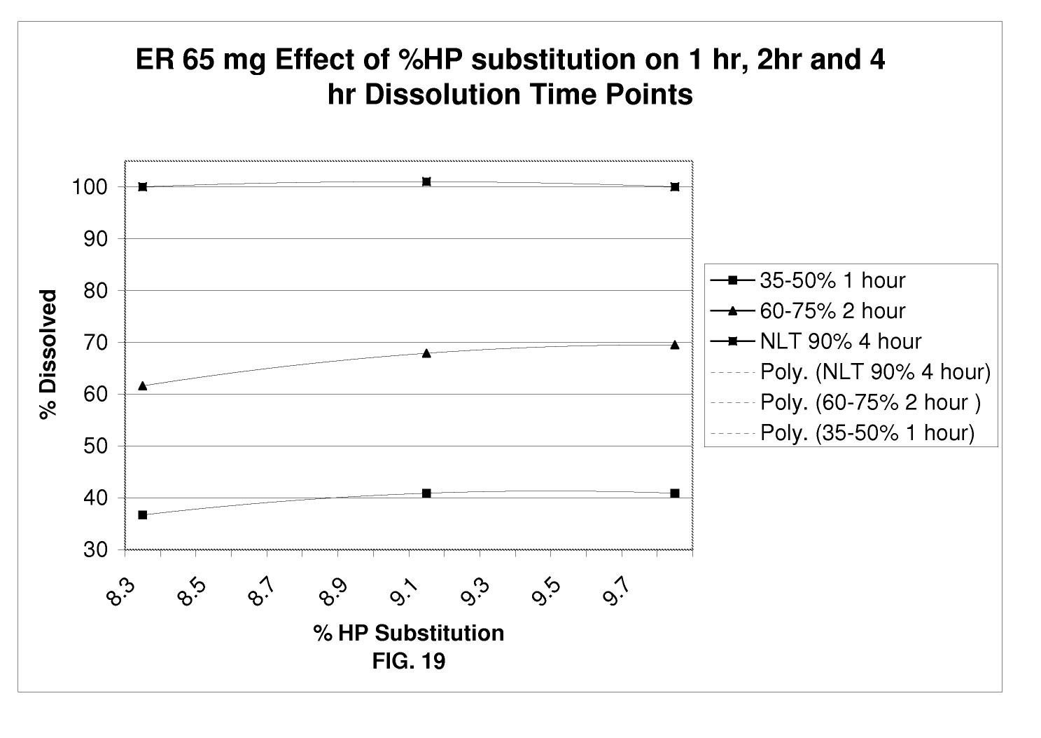

FIG. 19 is a comparison of dissolution profiles of 65 mg ER tablets according to the experimental data of Tables 32 (for 9.1 and 9.8% HP) and 43 (for 8.3% HP). Best-fit trend lines for 1-hour timepoints, 2-hour timepoints, and 4-hour timepoints are polynomial, showing that percent dissolution at a given timepoint increases nonlinearly.

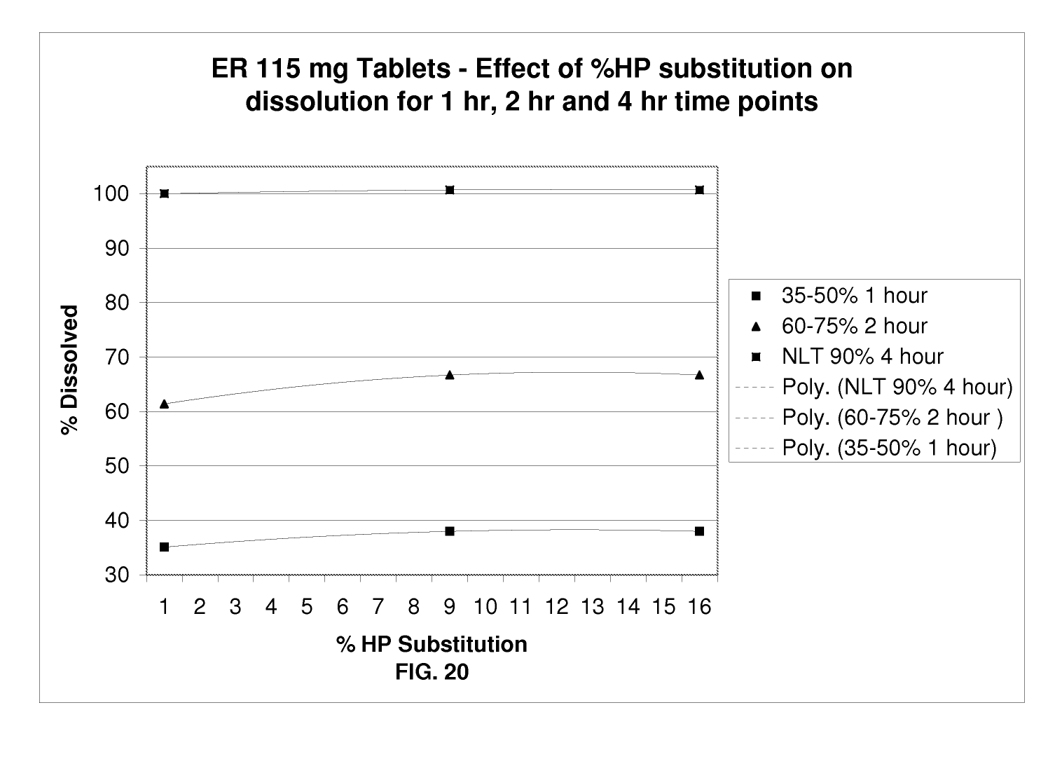

FIG. 20 is a comparison of dissolution profiles of 115 mg ER tablets according to the experimental data of Tables 37 (for 8.9 and 9.8% HP) and 39 (for 8.3% HP). Best-fit trend lines for 1-hour timepoints, 2-hour timepoints, and 4-hour timepoints are polynomial, showing that percent dissolution at a given timepoint increases nonlinearly.

Tablets that are the subject of the present disclosure are formulated to have an overall weight that is substantially constant across all strengths at 400 mg. Further, tablets that are the subject of the present disclosure are formulated to have consistent weight percentages of HPMC (27% for 45, 90, 115 mg, or 23.5% for 135 mg). Thus, a 115 mg strength tablet will have a higher ratio of active agent to HPMC than will a 45 mg strength tablet.

TABLE 50
Compositions that are measured to have
f2 > 80% as compared to NDA composition.

| Strength | HPMC, wt % | % HP HPMC | Active:HPMC |
|---|---|---|---|
| 45 | 27 | 8.4-8.7 | 11.25% |
| 65 | 27 | 8.3 | 16.25 |
| 90 | 27 | 8.4-8.7 | 22.50 |
| 115 | 27 | 8.3 | 28.75 |
| 135 | 23.5 | 8.3-8.7 | 33.75 |

According to test results for 45, 90 mg dosage forms at 8.4-8.7% HP, f2 is predicted to be >90. For the 135 mg dosage forms at 8.3-8.7% HP, f2 is predicted to be >90, and as high as >95.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A method of producing a batch of a tetracycline-class component HPMC extended release pharmaceutical product having a desired dissolution profile, comprising:
selecting a dissolution rate-controlling polymer comprising an HPMC component, the HPMC component having a selected % HP value;
validating that the % HP in the selected HPMC component is such that a mean sample of the product complies with the desired dissolution profile over each time point in the dissolution profile, and
preparing the product by preparing a formulation comprising a pharmaceutically effective amount of the tetracycline-class chemical and the selected HPMC component with the % HP value.

2. The method of claim 1, further comprising the steps of
subjecting a sample of batches to dissolution testing; and
selecting only those batches in which the mean dissolution profile for products from the batches has a similarity in terms of dissolution profile to the desired dissolution profile with an F2 similarity of 80% or greater.

3. The method of claim 1, wherein the dissolution rate-controlling polymer is present in an amount between 20 and 30 percent of the total weight of the product.

4. The method of claim 1, wherein the % HP is in a range between 8.3% and 9.8%.

5. The method of claim 1, wherein the tetracycline-class compound is selected from the group consisting of doxycycline, minocycline, and pharmaceutically acceptable salts and hydrates thereof.

6. The method of claim 1, wherein the product is a tablet.

7. The method of claim 1, wherein the plurality of dosage forms includes 45 mg, 90 mg and 135 mg dosage forms.

8. The method of claim 1, wherein the plurality of dosage forms includes 65 mg and 115 mg dosage forms.

9. The method of claim 1, wherein the tetracycline is minocycline hydrochloride.

10. The method of claim 1, wherein the % HP provides each of the plurality of dosage forms with the desired dissolution profile at 1 hr., 2 hr., and 4 hr time points.

11. The method of claim 1, wherein the % HP provides a number of batches of the product each having a different one of the plurality of dosages and each having the desired dissolution profile.

12. A method of achieving a desired dissolution profile for an extended release product for each one of a plurality of a dosage form, comprising:
selecting the product comprising an amount of a tetracycline-class compound and a dissolution rate-controlling polymer, the dissolution rate-controlling polymer having an HPMC component, the HPMC component having a selected % HP that controls dissolution so that the dissolution profile is met over each time point for the extended period of time for each one of the plurality of the dosage forms.

13. The method of claim 1, wherein the dissolution rate-controlling polymer is present in an amount between 20 and 30 percent of the total weight of the product.

14. The method of claim 13, wherein the selected % HP is in a range between 8.3% and 9.8%.

15. A method of predicting the desired dissolution profile within a desired accuracy in a first dosage form of a swellable matrix tablet having a dissolution rate-controlling polymer with an HPMC component, based on a second dosage form of the same swellable matrix tablet having the same dissolution rate-controlling polymer and the same HPMC component in the same amount, comprising
determining a % HP in the HPMC component of the second dosage form;
selecting the same % HP for the first dosage form; and
calculating, within a desired accuracy, the dissolution profile of the first dosage form based on the dissolution profile of the second dosage form.

16. The method of claim 15, wherein the dissolution profile has several time points for the extended period of time, and wherein the % HP can be raised or lowered in a range of 8.3% to 9.8% to raise or lower, respectively, the dissolution rate at one or more of the time points of the dissolution profile.

17. An extended release dosage form derived from a batch of a plurality of that dosage form comprising:
a core tablet comprising a tetracycline-class compound and a dissolution rate-controlling polymer, the dissolution rate-controlling polymer is between 20% and 30% of the total weight of the dosage form,
wherein the dissolution rate-controlling polymer has an HPMC component with a % HP that controls dissolution rate of the dosage form, and
wherein the % HP causes a consistent dissolution profile having a desired 1 hr., 2 hr., and 4 hr. dissolution rate that is consistent within the batch.

18. The dosage form of claim 17, wherein the tetracycline-class compound is present in a free base equivalent amount selected from the group consisting of 45, 65, 90, 110, 115, and 135 mg.

19. The dosage form of claim 17, wherein the tetracycline-class compound is selected from the group consisting of doxycycline, minocycline, and pharmaceutically-acceptable salts and hydrates thereof.

20. The dosage form of claim 1, wherein the % HP ranges from 8.3 to 9.8%.

21. A method of manufacturing an extended release dosage form that meets a required dissolution profile, comprising:
selecting a desired % HP in an HPMC component from a batch of HMPC; and
validating that the % HP selected is the desired % HP,
wherein the HMPC component is used in a product comprising an amount of a tetracycline-class compound and the HPMC component, and
wherein the % HP controls the dissolution profile for the extended release dosage form.

22. The method of claim 21, further comprising validating that the % HP achieves the dissolution profile within an f similarity of greater than 80%.
